(19) United States
(12) Patent Application Publication
(10) Pub. No.: US 2024/0417803 A1
(43) Pub. Date: Dec. 19, 2024

(54) METHOD AND KIT FOR DIAGNOSING TUMOURS

(71) Applicant: TECHNISCHE UNIVERSITÄT DRESDEN, Dresden (DE)

(72) Inventors: Mario MENSCHIKOWSKI, Dresden (DE); Markus FRIEDEMANN, Dresden (DE)

(73) Assignee: TECHNISCHE UNIVERSITÄT DRESDEN, Dresden (DE)

(21) Appl. No.: 18/701,886

(22) PCT Filed: Oct. 21, 2022
(86) PCT No.: PCT/EP2022/079395

§ 371 Date: Apr. 16, 2024

(30) Foreign Application Priority Data
Oct. 22, 2021 (DE) 10 2021 127 535.0

(51) Int. Cl. C12Q 1/6886; G16H 50/20

(52) U.S. Cl. CPC C12Q 1/6886; G16H 50/20; C12Q 2600/154; C12Q 2600/16

(57) ABSTRACT

The invention relates to a method for diagnosing a tumour disease in an isolated sample, comprising the amplification of methylated DNA sequences, to a kit for diagnosing a tumour disease, and to the use of the method and/or of the kit for the diagnosis and/or progress control of a malignant tumour disease, in particular of prostate, breast, ovarian or colorectal carcinomas. The invention furthermore relates to a computer program product comprising risk score analysis commands for diagnosing a tumour disease, and to a data processing device.

The invention relates to a method for diagnosing a tumour disease in an isolated sample, comprising the amplification of methylated DNA sequences, to a kit for diagnosing a tumour disease, and to the use of the method and/or of the kit for the diagnosis and/or progress control of a malignant tumour disease, in particular of prostate, breast, ovarian, or colorectal carcinomas. The invention furthermore relates to a computer program product comprising risk score analysis commands for diagnosing a tumour disease, and to a data processing device.

The time of diagnosis is crucial for the prognosis of cancers such as prostate and colorectal carcinomas. Thus, the 5- or 10-year survival rates depend greatly on the tumour stage in which the disease was discovered. Diagnosis of a tumour disease that is made too late is often associated with already occurring metastases. Therefore, for a timely diagnosis of malignant degenerations, biomarkers are required that detect such a degeneration with high diagnostic sensitivity and specificity.

The tumour markers known to date and used in clinical chemistry diagnostics have previously proven to be of value in particular in therapy control and aftercare of tumour patients. However, except for a few exceptions, they cannot be used in early diagnostics (screening) of tumour diseases. Here, diagnostic sensitivities and specificities are insufficient to reliably distinguish tumour patients from healthy individuals using these biomarkers, in particular at the early stage of development of the disease, without running the risk of overdiagnosis and overtherapy.

The diagnosis of prostate cancer is usually based on a digital rectal examination (DRE) and/or the measurement of the prostate-specific antigen (PSA) in serum (Lubolt et al. 2004). PSA is a serine protease secreted by the epithelial cells of the prostate in an organ-specific but not cancer-specific manner. Hence, increased PSA values also occur in non-malignant diseases, such as benign prostatic hyperplasia (BPH) and prostatitis (Lubolt et al. 2004). In general, the higher the PSA value, the more likely the presence of a prostate carcinoma (PCa). The European Association of Urology believes that men from the age of 40 years and a PSA>1 ng/ml and men from the age of 60 years and a PSA>2 ng/ml have an increased risk of advanced or metastatic prostate carcinoma (Lubolt et al. 2004). In order to detect risk patients at an early stage, a PSA screening, i.e., the systematic examination of symptom-free men, is discussed. About 85% of PCa are diagnosed with this test (Glass et al. 2013). However, a negative result does not reliably exclude the presence of a PCa. One major problem of PSA screening are the tissue biopsies that are many times indicated because of increased PSA values, but unnecessary. In Germany alone, approximately 200,000 to 300,000 biopsies are carried out annually due to unclear PSA value constellations; 65 to 70% thereof are subsequently found to be unnecessary as no tumour cells can be detected.

The diagnostic standard method in case of a positive PSA result and/or suspicious DRE result is a transrectal ultrasound scan (TRUS) (Lubolt et al. 2004). If there are no carcinoma cells in the biopsy, the current guidelines recommend a new biopsy within six months if the results are as follows: extensive high-grade PIN (found in at least 4 tissue samples), atypical small acinarproliferation (ASAP), isolated intraductal carcinoma of prostate (IDC-P), or if the PSA value or PSA profile remains suspicious. However, the biopsy is perceived as very unpleasant by most patients and is also associated with a non-insignificant risk of intervention. Prostate biopsy is an invasive diagnostic procedure, which in 1 to 2% of all cases is associated with side effects such as bleeding, inflammation and pain that need to be treated (Lubolt et al. 2004). This affects between 2000 and 6000 patients per year. Moreover, not all PCa are detected by a one-time tissue biopsy, which is why the removal of serial (up to ten) biopsies is carried out. The advantage of additional biopsies is controversial. Roehl et al. describe that 77% of the PCa were discovered when taking only one biopsy, whereas 99% of the tumours could be detected after four serial biopsies without thereby inducing an increase in the overdiagnosis of clinically irrelevant tumours (Roehl et al. 2002). This is contradicted by another study in which 63% of the tumours detected by tissue biopsies were clinically irrelevant and the additional diagnostic benefit of more than two biopsies is very low, which is why an extremely restrictive performance of serial biopsies was proposed for men who already had two negative prostate biopsy results (Zaytoun et al. 2012).

In contrast to prostate carcinomas, there is a suitable prevention or screening procedure for colonic carcinomas, namely the colonoscopy. However, the success of this screening is limited due to its relatively low acceptance by the population.

An alternative are novel faecal immunochemical occult blood tests (iFOBT), wherein a sensitivity of at most 25% and a specificity of at most 90% is achieved in the case of advanced adenomas, and thus only about half of the advanced adenomas identified by colonoscopy are detected by iFOBT (Tao et al. 2011).

In addition to sequence-dependent changes, tumour cells differ by sequence-independent epigenetic changes of the DNA, including hypermethylations. These tumour-specific changes in DNA methylation can be used as cancer markers (e.g., WO 2012/007462 A1, WO 2013/064163 A1, WO 2012/174256 A2, US 2011/0301050 A1).

However, the detection of these altered methylation patterns of selective DNA sections in blood, urine, or other bodily fluids has hitherto only been possible with insufficient analytical sensitivity. The degree of methylation of different target genes is determined primarily by means of PCR-based methods, which, with the exception of methylation-sensitive restriction enzyme analysis (MSRE-PCR), follow a bisulphite pre-treatment of genomic DNA. The detection limits of the individual methods for the detection of methylated DNA are different. Direct-BSP (sequencing according to Sanger) has a sensitivity of 10 to 20%, while pyrosequencing and MALDI-TOF mass spectrometry-based methods achieve sensitivities of about 5% (Mikeska et al. 2010, Kristensen and Hansen 2009). MSP (methylation-specific PCR), MethyLight, SMART-MSP (Sensitive Melting Analysis after Real Time-Methylation-Specific PCR) and MS-HRM (methylation-sensitive high-resolution melting) have a detection sensitivity of between 0.1% and 1.0% (Mikeska et al. 2010, Kristensen and Hansen 2009, Shen and Waterland 2007, Hernendez et al. 2013). One major disadvantage of previous PCR-based methods discussed is the so-called PCR bias, a phenomenon whereby methylated and non-methylated DNA strands are amplified with different efficiency.

Another problem of previous detection methods for DNA methylation are false-positive results which can arise due to an incomplete bisulphite conversion and non-specific primer annealing, in particular when using methyl-specific primers (Hernendez et al. 2013). Furthermore, none of the methods mentioned allows for detecting heterogeneously methylated DNA fragments, so-called epialleles, in a sensitive and above all quantitative manner. A new technique is digital PCR. With this technique, there is no PCR bias since each DNA molecule is amplified in a separate reaction compartment. Depending on the amount of DNA available and the assay design, sensitivities of up to 0.001% were described for dPCR. Another advantage of this technique is that absolute values are achieved even without using calibrators.

So far, a commercial test based on the detection of cellular epigenetic changes such as DNA methylation as an early and typical feature of malignant changes exists for the diagnosis of colorectal carcinoma with the determination of the methylated SEPT9 (Grützmann et al. 2008), of gliomas by MGMT determination, and lung carcinomas by SHOX2 determination (Shen and Waterland 2007). However, the sole determination of the methylated SEPT9 is associated with insufficient diagnostic sensitivity and specificity so that not all colorectal carcinomas are reliably detected by blood analysis.

For PCa, the LightMix Kit GSTP1 by the company Epigenomics is commercially available, but due to lack of high analytical sensitivity, this test has not hitherto been used in a broad clinical chemical application in the diagnosis of prostate carcinomas in conjunction with PSA determination.

In order to significantly improve the indication for colonoscopy and acceptance of these screening methods in the population, new biomarkers and analytical methods are also required which have a sensitivity significantly higher than 25% for colorectal carcinoma and, at the same time, show 100% diagnostic specificity.

DE 102015226843 B3 or U.S. Ser. No. 10/689,693 B2 describe the optimised bias-based pre-amplification digital droplet PCR (OBBPA-ddPCR) measuring method with the aid of which the biomarkers can be detected more sensitively and more specifically compared to other measuring methods, and unnecessary biopsies or colonoscopies can thus be avoided. In this method, freely circulating tumour DNA is determined as so-called “liquid biopsy” based on methylated sequences in patient samples such as serum, plasma, urine, liquor, stool, sputum, bronchoalveolar lavage, or sperm fluid.

Draht et al. describe prognostic DNA methylation markers for sporadic colorectal cancer, in particular RASSF1A or SEPT9 (Draht et al. 2018). WO 2019/068082 A1 discloses a method to detect a level of at least six preselected DNA methylation biomarkers, wherein the DNA methylation biomarkers, like miR129-2, CCDC181, GSTP1 or SEPT9, are used for cancer diagnosing, e.g. bladder urothelial carcinoma (BLCA), breast invasive carcinoma (BRCA) or pancreatic adenocarcinoma (PAAD).

The object of the invention is therefore to specify an improved method for diagnosing tumour diseases and a kit for the execution thereof, in particular for early diagnostics and for distinguishing benign tumours from malignant ones with increased sensitivity and specificity.

The object is achieved by the features of the independent claims. Advantageous embodiments are specified in the dependent claims.

According to the invention, novel biomarkers, in particular a combination of at least four genes selected from GSTP1, RASSF1A, CCDC181, NRIP3, miR129-2, SOX8, S1PR1, SYNE1, SFMBT2, ZNF304, or SEPTIN9, are provided which significantly increase diagnostic sensitivity and specificity for the diagnosis of tumour diseases, in particular in combination with PSA determination (total PSA [tPSA], free PSA [fPSA], or fPSA/tPSA quotient [QfPSA] and tPSA doubling time [tPSA-DT]), and thus make a decisive contribution to significantly reducing the number of unnecessary tissue biopsies.

According to the invention, a determination of the degree of methylation of the biomarkers (the sequences and genomic regions are shown in Table 1) is carried out using the method according to the invention and/or the kit according to the invention.

A first aspect of the invention relates to a method for diagnosing a tumour disease in an isolated sample, comprising the steps of:
a) isolating DNA from an isolated sample;
b) bisulphite conversion of the DNA, wherein a conversion of non-methylated cytosine residues into uracil residues takes place;
c) amplifying methylated DNA sequences of at least four genes selected from GSTP1, RASSF1A, CCDC181, NRIP3, miR129-2, SOX8, S1PR1, SYNE1, SFMBT2, ZNF304, or SEPTIN9 by means of PCR;
d) quantifying the amplified methylated DNA sequences by means of digital PCR.

According to the invention, the method according to the invention is carried out in the following sequence of steps: a), b), c) and d).

Surprisingly, it has been found that the genes (biomarkers) used in the method according to the invention differ significantly in terms of their methylation between blood samples from patients with prostate cancer and blood samples from healthy subjects.

The aforementioned abbreviations of the genes (biomarkers) are common designations. In addition, the genes used as biomarkers in the invention are defined by the Ensembl IDs shown in Table 1 and Table 3.

Advantageously, for the genes (biomarkers) RASSF1A, SOX8, miR129-2, GSTP1, CCDC181, and NRIP3 used in the method according to the invention, it was possible to determine in each case, with 100% diagnostic specificity, a sensitivity of 41.9% (RASSF1), 39.5% (SOX8), 65.1% (miR129-2), 34.9% (GSTP1), 32.6% (CCDC181), and 34.9% (NRIP3). Surprisingly, with a combination of the genes used (biomarkers) in the method according to the invention, it was possible to achieve a 76.7% sensitivity with 100% specificity or a 90.7% sensitivity with 79.3% specificity in patients with prostate carcinoma, a 100% sensitivity with 100% specificity in patients with colorectal carcinoma, a 77.8% sensitivity with 100% specificity in patients with breast carcinoma, and a 69.6% sensitivity with 100% specificity in patients with ovarian carcinoma. Advantageously, the method according to the invention thus has a higher sensitivity and specificity than known methods. In comparison, PSA determination results in a 91% diagnostic sensitivity with low specificity of 14 to 21% in patients with prostate carcinoma (Rashid et al. 2012).

In addition, a significant positive correlation between the degree of methylation of the genes (biomarkers) used according to the invention and the PSA value of PCa patients was determined. Advantageously, in the case of prostate cancer, the method according to the invention, in particular in combination with the determination of serum PSA, makes it possible to avoid unnecessary prostate tissue biopsies or to significantly increase the indication for a prostate carcinoma detection by biopsy in the case of normal and unremarkable PSA values. This reduces the number of false-positive and false-negative results. In this way, the number of annually performed prostate biopsies, which later turn out to be unnecessary and in Germany alone amount to between 160,000 and 230,000 biopsies per year, can be significantly reduced by a preceding blood test using the method according to the invention.

Furthermore, in the case of colorectal carcinoma, the acceptance of screening colonoscopy in the population and the diagnostic sensitivity and specificity of the examinations can advantageously be increased. In this way, the number of colorectal carcinomas that are detected too late and already have an advanced stage of development can be reduced, and the prognosis of a diagnosed disease can be significantly improved.

In embodiments, the DNA is free-circulating DNA fragments (fcDNA) or genomic DNA fragments.

In embodiments, the isolated sample is a tissue sample, a bodily fluid, a faecal sample, or a smear.

In embodiments, the isolated sample is a liquid biopsy sample. The term “liquid biopsy sample” is understood to mean a sample obtained by taking liquid from the body. The liquid biopsy is advantageously a minimally invasive sampling method. In embodiments, the isolated sample is whole blood, serum, plasma, urine, liquor (cerebrospinal fluid), sputum, bronchoalveolar lavage, sperm fluid, mammary gland secretion, vaginal fluid, or lymph, preferably serum, plasma, or urine.

The DNA isolation in step a) is carried out using methods known to the person skilled in the art. The performance of bisulphite conversion in step b) is also known to the person skilled in the art. For both steps, the person skilled in the art can use commercially available kits.

In embodiments, amplification of methylated and non-methylated DNA sequences of at least four genes selected from GSTP1, RASSF1A, CCDC181, NRIP3, miR129-2, SOX8, S1PR1, SYNE1, SFMBT2, ZNF304, or SEPTIN9 is carried out by means of PCR in step c), wherein the methylated DNA sequences and non-methylated DNA sequences are in each case amplified simultaneously with a primer pair, and a quantification of the amplified methylated and non-methylated DNA sequences by means of digital PCR is carried out in step d).

In step c), methylated and optionally corresponding non-methylated DNA sequences of the same gene segment are amplified by means of PCR. Expediently, a stronger amplification of the methylated DNA sequences (tumour-specific) than of the non-methylated DNA sequences takes place in step c).

In embodiments, the PCR in step c) is a bias-based PCR amplification (BBPA)-dPCR.

The term “bias” (distortion) is understood to mean a phenomenon whereby methylated and non-methylated DNA strands are amplified with different efficiency.

In embodiments, the primers and the PCR reaction conditions in step c) are selected such that they amplify both methylated and non-methylated DNA sequences, i.e., they are methylation-sensitive. Surprisingly, it has been found that compared to PCR with methylation-specific primers (MSP), the number of false-positive results is significantly reduced in the method according to the invention. Advantageously, heterogeneously methylated DNA (so-called epialleles) is also amplified in the method according to the invention.

It has been advantageously found that in the method according to the invention, the preferred amplification of methylated sequences is also possible in samples where there is high background DNA (non-methylated DNA). This DNA does not interfere with the method according to the invention. The method according to the invention is thus also suitable for the analysis of tumour DNA in bodily fluids (free-circulating DNA fragments, fcDNA).

The term “biomarker” is understood to mean a measurable parameter of biological processes, which has prognostic or diagnostic significance.

In embodiments, the following primer pairs are used in step c) for the amplification of methylated DNA sequences and optionally of the non-methylated DNA sequences of the genes mentioned:

TABLE 1
Primer sequences of the primer pairs for the method according to the
invention. The Ensembl ID, the coordinates of the plus strand of the
differentially methylated genomic regions (DMR) according to GRCh38/hg38,
and the sequences of the forward and reverse primers with the
respective SEQ ID nos. are listed for the genes (biomarkers).

| | | | Forward | SEQ | Reverse | SEQ |
|---|---|---|---|---|---|---|
| Gene | ID | DMR (+strand, | primer | ID | primer | ID |
| (biomarker) | Ensembl | GRCh38/hg38) | (5′→3′) | No. | (5′→3′) | No. |
| TMEM106A | ENSG0000 | chr17: | AGTTTTG | 1 | TCCGAAC | 2 |
| | 0184988 | 43.210.830- | ATTTAGT | | CCTCGAA | |
| | | 43.212.830 | GTTTAGG | | TAAC | |
| | | | GA | | | |
| EYA4 | ENSG0000 | chr6: | GGCGTTA | 3 | AAAACCC | 4 |
| | 0112319 | 133.240.082- | TTCGGGA | | ACTCCGT | |
| | | 133.242.082 | TATTTA | | TTTT | |
| GRIA4 | ENSG0000 | chr11: | GGATGAG | 5 | CCGAACT | 6 |
| | 0152578 | 105.609.653- | ATATGCG | | AATACAA | |
| | | 105.611.653 | TTTTATTT | | ACTTACT | |
| | | | T | | AA | |
| CCDC181 | ENSG0000 | chr1: | TCGTTTT | 7 | CCTCCTC | 8 |
| | 0117477 | 169.426.454- | CGTAGTT | | TTTCTAA | |
| | | 169.428.454 | AGAGAG | | ACTCTTC | |
| | | | GTT | | ACA | |
| CCDC181 | ENSG0000 | chr1: | TGGAGGA | 9 | CCTCCTC | 10 |
| | 0117477 | 169.426.454- | CGTAGAG | | TTTCTAA | |
| | | 169.428.454 | GTACGTT | | ACTCTTC | |
| | | | G | | ACATTA | |
| CCDC181 | ENSG0000 | chr1: | CGTAGTT | 11 | CCTCCTC | 12 |
| | 0117477 | 169.426.454- | AGAGAG | | TTTCTAA | |
| | | 169.428.454 | GTTCGGA | | ACTCTTC | |
| | | | TGG | | ACATTA | |
| CCDC181 | ENSG0000 | chr1: | CGTAGTT | 13 | CTCCTCT | 14 |
| | 0117477 | 169.426.454- | AGAGAG | | CCTCCTC | |
| | | 169.428.454 | GTTCGGA | | TTTCTAA | |
| | | | TG | | AC | |
| CCDC181 | ENSG0000 | chr1: | CGGATGG | 15 | CTCCTCT | 16 |
| | 0117477 | 169.426.454- | AGGACG | | CCTCCTC | |
| | | 169.428.454 | CAGAGG | | TTTCTAA | |
| | | | CACG | | AC | |
| CCDC181 | ENSG0000 | chr1: | CGGATGG | 17 | CTCCTCT | 18 |
| | 0117477 | 169.426.454- | AGGACG | | CCTCCTC | |
| | | 169.428.454 | CAGAGG | | TTTCTAA | |
| | | | CAC | | AC | |
| ADAM32 | ENSG0000 | chr8: | GTATTTTA | 19 | AAATCCA | 20 |
| | 0197140 | 39.106.540- | GTCGCGT | | ACAACAC | |
| | | 39.108.540 | GGTTG | | AAAACGA | |
| RASSF1A | ENSG0000 | chr3: | GTTTGTT | 21 | AATACGA | 22 |
| | 0068028 | 50.339.829- | AGCGTTT | | CCCTTCC | |
| | | 50.341.829 | AAAGTTA | | CAAC | |
| | | | G | | | |
| GSTP1 | ENSG0000 | chr11: | ATCGTAG | 23 | TCCCCAA | 24 |
| | 0084207 | 67.582.477- | CGGTTTT | | CGAAACC | |
| | | 67.584.477 | AGGGAAT | | TAAAAA | |
| VWA3B | ENSG0000 | chr2: | CGCGGG | 25 | CATCCCG | 26 |
| | 0168658 | 98.086.172- | TTATAGA | | CTAATAA | |
| | | 98.088.172 | GATTTAG | | ATAAAAC | |
| | | | | | AA | |
| NRIP3 | ENSG0000 | chr11: | TTACGTT | 27 | ATAACAA | 28 |
| | 0175352 | 9.003.264- | AGTTTAG | | AACCGAC | |
| | | 9.005.264 | GGGATG | | GAAAAA | |
| NRIP3 | ENSG0000 | chr11: | TTAGTTTA | 29 | CTCACCC | 30 |
| | 0175352 | 9.003.264- | GGGGAT | | GATAACA | |
| | | 9.005.264 | GCGG | | AAACC | |
| NRIP3 | ENSG0000 | chr11: | CGGCGT | 31 | CCCTAAA | 32 |
| | 0175352 | 9.003.264- | GGATAGG | | CTAACGT | |
| | | 9.005.264 | TGAGTAG | | AAACTCT | |
| | | | | | AAA | |
| NRIP3 | ENSG0000 | chr11: | CGGCGT | 33 | CCCTAAA | 34 |
| | 0175352 | 9.003.264- | GGATAGG | | CTAACGT | |
| | | 9.005.264 | TGAGT | | AAACTCT | |
| | | | | | AAA | |
| NRIP3 | ENSG0000 | chr11: | CGTGGAT | 35 | CCCTAAA | 36 |
| | 0175352 | 9.003.264- | AGGTGAG | | CTAACGT | |
| | | 9.005.264 | TAGG | | AAACTCT | |
| | | | | | AAA | |
| ZNF833 | ENSG0000 | chr19: | CGTTTGT | 37 | ATTAAATA | 38 |
| | 0267343 | 11.672.857- | TAAGGGT | | CGACTCC | |
| | | 11.674.857 | TGTGTTT | | AAATCCC | |
| | | | G | | G | |
| SOX8 | ENSG0000 | chr16: | GTAGGTT | 39 | TAACTCG | 40 |
| | 0005513 | 979.544- | ATTCGGG | | AATCCTT | |
| | | 981.544 | TTTCG | | TCAACC | |
| SOX8 | ENSG0000 | chr16: | GGGTCGT | 41 | AATAACT | 42 |
| | 0005513 | 979.544- | AAGTTCG | | CGAATCC | |
| | | 981.544 | GAGA | | TTTCAAC | |
| | | | | | CA | |
| ZNF529 | ENSG0000 | chr19: | TTTTAGG | 43 | TAATACC | 44 |
| | 0186020 | 36.604.267- | AGTTTTC | | AACCGCA | |
| | | 36.606.267 | GGGG | | ATAAAAC | |
| miR129-2 | ENSG0000 | chr11: | ACGGTTT | 45 | CGAACCC | 46 |
| | 0199077 | 43.580.221- | GGAGAAA | | TAAAACC | |
| | | 43.582.221 | TGGAGAT | | GAACAAA | |
| USP44 | ENSG0000 | chr12: | GAGAATT | 47 | ACACTAC | 48 |
| | 0136014 | 95.547.931- | TGTCGGT | | AAATACG | |
| | | 95.549.931 | GGTC | | AAACCC | |
| HES5 | 0197921 | 2.531.343 | CGGTTAA | 49 | AAAACGC | 50 |
| | ENSG0000 | chr1: | TGGT | | CCATTATA | |
| | | 2.529.343- | ATATTTCG | | CCC | |
| ZFP37 | ENSG0000 | chr9: | TTTTGGG | 51 | ATCAATC | 52 |
| | 0136866 | 113.055.811- | ATAATTAG | | CTATAATA | |
| | | 113.057.811 | ATCGGG | | CGTAACG | |
| | | | | | A | |
| PCSK9 | ENSG0000 | chr1: | GTCGTAC | 53 | AATCGCC | 54 |
| | 0169174 | 55.039.262- | GTTTTAG | | GAAAACT | |
| | | 55.041.262 | TAAGTTT | | | |
| | | | TT | | CTTC | |
| RNF39 | ENSG0000 | chr6: | GTATATC | 55 | AACGTCC | 56 |
| | 0204618 | 30.070.643- | GTCGTTT | | CTAATAA | |
| | | 30.072.643 | GTTTATTT | | AACCA | |
| | | | T | | | |
| RNF39 | ENSG0000 | chr6:3 | GTCGTAG | 57 | GCACCCA | 58 |
| | 0204618 | 0.070.643- | CGTATAAT | | ACACAAC | |
| | | 30.072.643 | TGGTT | | TAAA | |
| VCY | ENSG0000 | chrY: | TTTATATT | 59 | CCCCGAA | 60 |
| | 0129864 | 13.750.425- | TGGACG | | CACTCAA | |
| | | 13.752.425 | GAGAGG | | CTAAA | |
| | | | G | | | |
| VCY | ENSG0000 | chrY: | CGTGGGT | 61 | CCCGAAC | 62 |
| | 0129864 | 13.750.425- | TTGTTTAT | | ACTCAAC | |
| | | 13.752.425 | ATTTGG | | TAAA | |
| VCY | ENSG0000 | chrY: | TGTTTATA | 63 | AAACCCC | 64 |
| | 0129864 | 13.750.425- | TTTGGAC | | GAACACT | |
| | | 13.752.425 | GGAGAG | | CAAC | |
| | | | G | | | |
| STOM | ENSG0000 | chr9: | TTGAGGT | 65 | ACGAAAT | 66 |
| | 0148175 | 121.368.936- | TACGATG | | AATACAA | |
| | | | | | CCCTAAC | |
| | | 121.370.936 | GAGG | | AT | |
| H2BC3 | ENSG0000 | chr6: | GGCGTTA | 67 | ACTTTAA | 68 |
| | 0276410 | 26.042.940- | TTTTAGTT | | CTCCATT | |
| | | 26.044.940 | TGTTTGT | | TTCCACG | |
| | | | T | | | |
| S1PR1 | ENSG0000 | chr1: | GGGTAGT | 69 | CCTACCT | 70 |
| | 0170989 | 101.236.025- | AGTAAGA | | CGCTCAA | |
| | | 101.238.025 | TGCGAAG | | ACAAA | |
| SYNE1 | ENSG0000 | chr6: | GGCGAG | 71 | CGCCCTA | 72 |
| | 0131018 | 152.635.869- | TTAGTTA | | ATAACAA | |
| | | 152.637.869 | GGGTGA | | AAAACAA | |
| | | | | | CA | |
| LONRF2 | ENSG0000 | chr2: | GAGTTTC | 73 | ACCTAAA | 74 |
| | 0170500 | 100.321.300- | GTAGGGT | | TTACCTA | |
| | | 100.323.300 | TGTAAT | | AACCGAC | |
| ZNF304 | ENSG0000 | chr19: | CGTTTTT | 75 | GATCTCT | 76 |
| | 0131845 | 57.350.216- | TGTTGTT | | AAAACTC | |
| | | 57.352.216 | TTGGGTT | | AACCTTC | |
| | | | | | AC | |
| ZNF304 | ENSG0000 | chr19: | TTGTAGT | 77 | GTTACCC | 78 |
| | 0131845 | 57.350.216- | TTAGAGT | | AAAAATC | |
| | | 57.352.216 | AGTAGGA | | TATACCC | |
| | | | AACGTT | | AACTC | |
| AKR1B1 | ENSG0000 | chr7: | TTTTTCG | 79 | GCTTTCC | 80 |
| | 0085662 | 134.458.249- | TTTGGTT | | CACCAAA | |
| | | 134.460.249 | TTAGTGG | | TACAA | |
| HSPA1A | ENSG0000 | chr6: | TTCGAAA | 81 | CGACTTA | 82 |
| | 0204389 | 31.814.570- | AAGGTAG | | TAAAAAC | |
| | | 31.816.570 | TGGATTG | | CCAAAAA | |
| | | | | | CA | |
| ZNF655 | ENSG0000 | chr7: | GGAAGC | 83 | GCCAACC | 84 |
| | 0197343 | 99.557.250- | GGGATTA | | CATCCTT | |
| | | 99.559.250 | TTAGGT | | AAAAC | |
| SFMBT2 | ENSG0000 | chr10: | GAGGGTA | 85 | TACTCTC | 86 |
| | 0198879 | 7.409.861- | GGACGAA | | CGACCAA | |
| | | 7.411.861 | TTTTATTA | | CCTA | |
| | | | G | | | |
| SFMBT2 | ENSG0000 | chr10: | GGGTAG | 87 | TAACCAA | 88 |
| | 0198879 | 7.409.861- | GACGAAT | | CGCACTC | |
| | | 7.411.861 | TTTATTAG | | AACC | |
| | | | GT | | | |
| ZNF543 | ENSG0000 | chr19: | GTGGGAA | 89 | CCTCCGA | 90 |
| | 0178229 | 57.319.312- | TTTATTAA | | CTTATAC | |
| | | 57.321.312 | GCGTAGT | | CCATAAT | |
| GNE | ENSG0000 | chr9: | GCGGTAG | 91 | AATTAAC | 92 |
| | 0159921 | 36.257.633- | AGGTTAT | | TACTCCC | |
| | | 36.259.633 | GGAA | | GTCCC | |
| SEPTIN9 | ENSG0000 | chr17: | GGAGTTG | 93 | ACTAACA | 194 |
| | 0184640 | 77.371.771- | GTGGTTT | | AACAAAA | |
| | | 77.374.542 | TTCGTTG | | TCCCGAC | |
| | | | G | | CT | |
| SEPTIN9 | ENSG0000 | chr17: | GTTTGTA | 95 | CCTCGAC | 96 |
| | 0184640 | 77.371.771- | AGGGTTA | | TCTTAAA | |
| | | 77.374.542 | AACGGTA | | CCCAC | |
| | | | G | | | |
| SEPTIN9 | ENSG0000 | chr17: | GGATTTC | 97 | CAAAATC | 98 |
| | 0184640 | 77.371.771- | GAAGGTG | | CTCTCCA | |
| | | 77.374.542 | GGTGT | | ACACGTC | |

In embodiments, step c) involves amplification of methylated and optionally non-methylated DNA sequences of the genes
a. GSTP1, RASSF1A, CCDC181, NRIP3, miR129-2, and SOX8, or
b. SIPR1, SYNE1, CCDC181, SFMBT2, ZNF304, and SEPTIN9, or
c. GSTP1, RASSF1A, CCDC181, and miR129-2.

In embodiments, step c) involves amplification of methylated and optionally non-methylated DNA sequences of the genes
a. GSTP1, RASSF1A, CCDC181, NRIP3, miR129-2, and SOX8 for the diagnosis of prostate carcinomas, or
b. S1PR1, SYNE1, CCDC181, SFMBT2, ZNF304, and SEPTIN9 for the diagnosis of colorectal carcinomas, or
c. GSTP1, RASSF1A, CCDC181, and miR129-2 for the diagnosis of breast and ovarian carcinomas.

In embodiments, the primer pair for amplification of methylated and optionally non-methylated DNA sequences in step c) is selected from
SEQ ID No. 23 and SEQ ID No. 24 for GSTP1,
SEQ ID No. 21 and SEQ ID No. 22 for RASSF1A,
SEQ ID No. 7 and SEQ ID No. 8 or SEQ ID No. 9 and SEQ ID No. 10 or SEQ ID No. 11 and SEQ ID No. 12 or SEQ ID No. 13 and SEQ ID No. 14 or SEQ ID No. 15 and SEQ ID No. 16 or SEQ ID No. 17 and SEQ ID No. 18 for CCDC181,
SEQ ID No. 27 and SEQ ID No. 28 or SEQ ID No. 29 and SEQ ID No. 30 or SEQ ID No. 31 and SEQ ID No. 32 or SEQ ID No. 33 and SEQ ID No. 34 or SEQ ID No. 35 and SEQ ID No. 36 for NRIP3,
SEQ ID No. 45 and SEQ ID No. 46 for miR129-2,
SEQ ID No. 39 and SEQ ID No. 40 or SEQ ID No. 41 and SEQ ID No. 42 for SOX8,
SEQ ID No. 69 and SEQ ID No. 70 for S1PR1,
SEQ ID No. 71 and SEQ ID No. 72 for SYNE1,
SEQ ID No. 85 and SEQ ID No. 86 or SEQ ID No. 87 and SEQ ID No. 88 for SFMBT2,
SEQ ID No. 75 and SEQ ID No. 76 or SEQ ID No. 77 and SEQ ID No. 78 for ZNF304, and
SEQ ID No. 93 and SEQ ID No. 94 or SEQ ID No. 95 and SEQ ID No. 96 or SEQ ID No. 97 and SEQ ID No. 98 for SEPTIN9.

In embodiments, step c) furthermore involves an amplification of methylated and optionally non-methylated DNA sequences of at least one gene selected from TMEM106A, EYA4, GR/A4, ADAM32, VWA3B, ZNF833, ZNF529, USP44, HES5, ZFP37, PCSK9, RNF39, VCY, STOM, H2BC3, LONRF2, AKR1B1, HSPA1A, ZNF655, ZNF543, or GNE, wherein a primer pair is used in each case to amplify the methylated DNA sequences and non-methylated DNA sequences simultaneously, and step d) furthermore involves a quantification of the amplified methylated DNA sequences and optionally the amplified non-methylated DNA sequences of the at least one gene by means of digital PCR.

In embodiments, the primer pair for amplification of methylated and optionally non-methylated DNA sequences in step c) is selected from
SEQ ID No. 1 and SEQ ID No. 2 for TMEM106A,
SEQ ID No. 3 and SEQ ID No. 4 for EYA4,
SEQ ID No. 5 and SEQ ID No. 6 for GRIA4,
SEQ ID No. 19 and SEQ ID No. 20 for ADAM32,
SEQ ID No. 25 and SEQ ID No. 26 for VWA3B,
SEQ ID No. 37 and SEQ ID No. 38 for ZNF833,
SEQ ID No. 43 and SEQ ID No. 44 for ZNF529,
SEQ ID No. 47 and SEQ ID No. 48 for USP44,
SEQ ID No. 49 and SEQ ID No. 50 for HES5,
SEQ ID No. 51 and SEQ ID No. 52 for ZFP37,
SEQ ID No. 53 and SEQ ID No. 54 for PCSK9,
SEQ ID No. 55 and SEQ ID No. 56 or SEQ ID No. 57 and SEQ ID No. 58 for RNF39,
SEQ ID No. 59 and SEQ ID No. 60 or SEQ ID No. 61 and SEQ ID No. 62 or SEQ ID No. 63 and SEQ ID No. 64 for VCY,
SEQ ID No. 65 and SEQ ID No. 66 for STOM,
SEQ ID No. 67 and SEQ ID No. 68 for H2BC3,
SEQ ID No. 73 and SEQ ID No. 74 for LONRF2,
SEQ ID No. 79 and SEQ ID No. 80 for AKR1B1,
SEQ ID No. 81 and SEQ ID No. 82 for HSPA1A,
SEQ ID No. 83 and SEQ ID No. 84 for ZNF655,
SEQ ID No. 89 and SEQ ID No. 90 for ZNF543, and
SEQ ID No. 91 and SEQ ID No. 92 for GNE.

In embodiments, DNA sequences of four to ten genes, preferably four to six genes, are analysed for the degree of methylation in the method according to the invention. For this purpose, step c) is preferably carried out as multiplex PCR, i.e., the primer pairs for amplification of the DNA sequences are matched to one another such that they have an annealing temperature in the same order of magnitude and do not hybridise to one another.

Preferably, the PCR conditions, in particular primer sequences, magnesium chloride concentration, and annealing temperature, are set such that the bias is optimised in favour of the methylated DNA sequences, i.e., so that primarily methylated DNA sequences are amplified. In the absence of methylated DNA sequences, non-methylated DNA sequences are amplified, which is advantageously used as an internal control reaction. An optimisation of the PCR reaction conditions advantageously enables a PCR bias of at least 80%, preferably in the range of 80% to 90%, in favour of the amplification of tumour DNA sequences. An optimisation of the PCR reaction conditions advantageously enables a sensitivity and specificity of up to 100%.

In embodiments, the PCR in step c) is carried out with a magnesium chloride concentration (final concentration) in the range from 0.5 mmol/I to 15 mmol/I, preferably in the range from 2 mmol/I to 5 mmol/I, particularly preferably in the range from 2.5 mmol/I to 3.5 mmol/1.

In embodiments, the primer pairs in step c) each have one to seven 5′-CG-3′ dinucleotides, preferably one to four 5′-CG-3′ dinucleotides, particularly preferably one to three 5′-CG-3′ dinucleotides.

In embodiments, at least two (different) primer pairs are used per biomarker or gene in the amplification according to step c). Surprisingly, it has also been shown that the diagnostic sensitivity of the method is further increased, i.e., the number of pathological results in actual tumour diseases increases, if at least two (different) primer pairs are used separately or simultaneously per biomarker or gene in the amplification according to step c), which primer pairs preferably include all DNA sequences quantified by probes in step c).

In embodiments, the annealing temperature is at least 40° C. In preferred embodiments, the annealing temperature is in the range between 50° C. and 72° C., preferably 53° C. to 70° C., particularly preferably 53° C. to 63° C.

Expediently, the optimal PCR conditions, in particular the annealing temperatures and/or magnesium chloride concentrations, are determined empirically for each gene (biomarker) and each primer pair, and the biases are optimised in favour of the methylated DNA sequences. Preferably, determination of the optimal PCR conditions takes place with at least one sample with a known methylated DNA/non-methylated DNA ratio, in particular with a fully methylated and a fully non-methylated DNA sequence of the gene (biomarker).

The number of PCR cycles in step c) depends on the starting concentration of the DNA in the isolated sample. In embodiments, cycle numbers in the range from 5 to 50, preferably in the range from 10 to 50, particularly preferably in the range from 12 to 40, are selected. Expediently, an increase in the number of cycles in step c) makes it possible to increase the stringency in the distinction between healthy and sick. This is particularly important when differentiating between benign hyperplasias, e.g., benign prostatic hyperplasia (BPH), and malignant diseases such as prostate carcinoma.

In embodiments, the methylated and optionally non-methylated DNA sequences are amplified in step c) by means of a correspondingly high number of PCR cycles, preferably 10 to 50 cycles. In embodiments, quantification according to step d) is subsequently carried out directly or after a slight pre-dilution of the amplicons in the case of a high number of cycles in dPCR.

In step d), a quantification is carried out by means of digital PCR (dPCR). In embodiments of digital PCR, a limiting dilution of the DNA used is carried out such that no or precisely one DNA molecule is present in a maximum number of compartments (Poisson distribution).

In alternative embodiments, the amount of DNA used in dPCR is increased beyond the Poisson distribution (e.g., in the case of 10,000 compartments, more than 80,000 DNA copies are analysed in dPCR, i.e., with a CPC [copy per compartment] value>8), thereby improving the specificity and differentiation between healthy and malignant tumour disease. According to the prior art, a Poisson distribution is present for dPCR if the CPC value is <8, since otherwise no compartments without DNA copies are present as a basis for the calculations.

By combining steps c) and d), also BBPA-dPCR, significantly higher sensitivities are advantageously achieved, on the one hand; on the other hand, the method according to the invention surprisingly allows for a much more reliable statement as to whether or not a malignant tumour exists. The method is thus suitable for early diagnosis screening. Another advantage of the method according to the invention is that it allows for distinguishing between benign and malignant tumours.

Digital PCR (dPCR) within the meaning of the invention is understood to mean a PCR in a large number of separate compartments, preferably with a volume in the femtolitre or nano range. dPCR is characterised in that the quantification of the compartments takes place after a digital result (amplification: yes or no). Statistical significance is achieved by counting a large number of reaction compartments (high-throughput screening with preferably 10,000 to 100,000 compartments per PCR). The percentage of reaction spaces with successful amplification is proportional to the used DNA amount of the amplified DNA sequence, which is used for quantification.

In embodiments, quantification of the amplified DNA by means of digital PCR in step d) takes place by means of hydrolysis probes.

In embodiments, the probes for quantifying the amplified methylated DNA sequences each have two to eight 5′-CG-3′ or CpG dinucleotides, preferably three to six 5′-CG-3′ dinucleotides in each case.

In embodiments, the probes for quantifying the non-methylated DNA sequences each have two to eight 5′-CA-3′ or 5′-TG-3′ dinucleotides, preferably three to six 5′-CA-3′ or 5′-TG-3′ dinucleotides in each case.

In further embodiments, quantification in step d) takes place by means of probes for the complementary DNA strand in the amplified DNA double-strand molecule. In embodiments, the complementary DNA strand is quantified separately or together with probes for the leading strand or lagging strand.

In embodiments, the number of 5′-CG-3′ dinucleotides in the probes for the methylated DNA sequences corresponds to the number of 5′-CA-3′ dinucleotides or 5′-TG-3′ dinucleotides in the probes for the non-methylated DNA sequences of the same gene (biomarker).

In embodiments, the number of 5′-CG-3′, 5′-CA-3′, or 5′-TG-3′ dinucleotides per gene (biomarker) is either contained in a probe or distributed among multiple probes, in particular if it is impossible due to the sequence of the gene to design a probe that comprises all methylation sites.

In embodiments, at least two probes are used in step d) which comprise different sequence sections in the amplicons generated during amplification. The diagnostic sensitivity of the method is thereby advantageously increased.

In embodiments, two probes are used for one gene, wherein both probes each contain three or four 5′-CG-3′ dinucleotides for the methylated DNA sequences, and both probes each contain three or four 5′-CA-3′ or 5′-TG-3′ dinucleotides for the non-methylated sequences.

In embodiments, the probes are fluorescently labelled. In further embodiments, the probes have different fluorescent markers for methylated and non-methylated DNA sequences. In embodiments, the fluorescent labelling is attached to one end of the probe, preferably at the 5′ end of the probe.

In preferred embodiments, the probes are labelled 5′-FAM (6-carboxyfluorescein) for the methylated DNA sequences, and 5′-HEX (hexachloro-fluorescein) for the non-methylated DNA sequences.

In preferred embodiments, quantification of the amplified DNA by means of digital PCR in step d) takes place by means of probes which have fluorescent markers and quenchers. The probe expediently has a quencher matching the fluorescent label, which quencher suppresses the fluorescence signal. In preferred embodiments, the quencher matching the fluorescent label is located at the other end of the probe, preferably at the 3′ end of the probe. By hybridisation or after hybridisation and subsequent polymerase action with the amplified DNA, the quenching effect is eliminated and the fluorescence signal can be detected. Such fluorescent labels and quenchers are well known to the person skilled in the art and are commercially available for any desired probe sequences.

In preferred embodiments, the probes are marked with the quencher BHQ-1 (Black Hole Quencher 1, 3′) at the 3′ end.

In embodiments, digital PCR is performed in step d) as multiplex PCR, using multicolour fluorescence detection systems. For quantification, differently fluorescently labelled probes are preferably used for each amplified gene (biomarker). The probes are expediently constructed to have a similar hybridisation temperature.

In alternative embodiments, the probes are used and evaluated in separate batches in dPCR, in particular if the probes have different hybridisation temperatures.

In further embodiments, a plurality of probes is used per gene (biomarker), wherein multicolour fluorescence detection systems are used or identical fluorescent markers are used and the obtained fluorescence signal is integrated or the probes with identical fluorescent markers are used and evaluated in separate batches.

In embodiments, the primer pairs used for amplification in step c) are used simultaneously in step d) during digital PCR.

In further embodiments, nested primers that bind at other sites of the amplified DNA sequences are used in step d). The term “nested polymerase chain reaction (nested PCR)” is understood to mean a modification of the polymerase chain reaction in which non-specific binding in the products is reduced due to the amplification of unexpected primer binding sites by using two sets of primers in two successive runs of the polymerase chain reaction, wherein the second set is intended to amplify a secondary target within the product of the first run. Advantageously, “nested” PCR increases specificity. In embodiments, the primers for “nested” PCR are selected by methods known to the person skilled in the art. Preferably, the primers for “nested” PCR are selected according to the above-described embodiments for step c).

The following reaction conditions are preferably used for the amplification of methylated and optionally non-methylated DNA sequences in step c) and the quantification in step d):

TABLE 2
PCR conditions in step c) and step d) for the corresponding
genes (biomarkers): magnesium chloride concentrations (MgCl₂) and
annealing temperatures (T_{A}). Hexaplex (a) represents the preferred
PCR conditions of a multiplex PCR of CCDC181, NRIP3, miR129-2,
RASSF1A, and GSTP1 and pentaplex (b) represents the preferred
PCR conditions of a multiplex PCR of CCDC181, S1PR1, SYNE1,
ZNF304, SFMBT2 (see last column).

| | | |
|---|---|---|
| Gene | Step c) | Step d) |

| (biomarker) | MgCl₂ | T_{A} | T_{A} | Multiplex PCR |
|---|---|---|---|---|
| CCDC181 | 2.5 | 60.4 | 61 | a, b |
| NRIP3 | 3.5 | 52.6 | 51.9 | a |
| miR129-2 | 2.5 | 63 | 53.8 | a |
| SOX8 | 3.5 | 52.6 | 50 | a |
| S1PR1 | 4.5 | 60.8 | 50 | b |
| SYNE1 | 3.5 | 60.8 | 50 | b |
| ZNF304 | 3.5 | 60.8 | 61 | b |
| SFMBT2 | 3.5 | 60.8 | 50 | b |
| RASSF1A | 4.5 | 59.0 | 50.7 | a |
| GSTP1 | 4.5 | 53.8 | 50.7 | a |
| TMEM106A | 3.5 | 52.5 | 50.7 | — |
| EYA4 | 4.5 | 52.5 | 51.9 | — |
| HES5 | 8 | 50.8 | 53.8 | — |
| SEPTIN9 | 3.5 | 58.0 | 50.7 | — |
| Hexaplex (a) | 3.5 | 58.0 | | |
| Pentaplex (b) | 3.5 | 60.8 | | |

Probes comprising the following sequences are preferably used for the quantification in step d):

TABLE 3
Probe sequences for the corresponding genes (biomarkers): The
Ensembl ID, the sequences of the fully methylated and fully
non-methylated probe, and the SEQ ID nos. are listed
for the genes (biomarkers).

| | | | SEQ | Non- | SEQ |
|---|---|---|---|---|---|
| Gene | | Methylated | ID | methylated | ID |
| (biomarker) | Ensembl ID | (5′→3′) | No. | (5′→3′) | No. |
| TMEM106A | ENSG00000184988 | AACGTCTCAA | 99 | AACATCTCAA | 100 |
| | | ACTACTACGT | | ACTACTACAT | |
| | | AACGAAA | | AACAAAA | |
| EYA4 | ENSG00000112319 | GCCCAAAAC | 101 | CATAAAAACC | 102 |
| | | CGTAAAAACC | | ACCCAAAAC | |
| | | CTCG | | CTCA | |
| CCDC181 | ENSG00000117477 | CCCGACACC | 103 | CCCAACACC | 104 |
| | | TACCTCCGC | | TACCTCCACA | |
| | | GACAACCAA | | ACAACCAA | |
| RASSF1A | ENSG00000068028 | AATACCAACT | 105 | CACCCAACA | 106 |
| | | CGCCCAACG | | AATACCAACT | |
| | | CCCG | | CCCA | |
| GSTP1 | ENSG00000084207 | ATATACGCAA | 107 | CACAACAAAA | 108 |
| | | CGCAACGAA | | TATACACAAC | |
| | | C | | | |
| NRIP3 | ENSG00000175352 | CGACAATCC | 109 | CAACCATCTC | 110 |
| | | GAAAATCCAC | | TACCACTAAC | |
| | | CG | | CA | |
| SOX8 | ENSG00000005513 | GTTGGTTGTT | 111 | TGTGTTTTGT | 112 |
| | | CGTGTTTCGC | | GTTGGTTGTT | |
| | | T | | TGATT | |
| SOX8 | ENSG00000005513 | GTTGGTTGTT | 113 | TGTGTTTTGT | 114 |
| | | CGTGTTTCGC | | GTTGGTTGTT | |
| | | T | | TGATT | |
| miR129-2 | ENSG00000199077 | CGACTCAATC | 115 | CATTCCATCA | 116 |
| | | CGTTCCATCG | | CAACTCAATC | |
| | | TCACCAC | | TCACCAC | |
| HES5 | ENSG00000197921 | TACGCTAATA | 117 | CAATTTAACA | 118 |
| | | CAATTTAACG | | TACACTAATA | |
| | | AACCGACG | | AACCAACA | |
| VCY | ENSG00000129864 | CCCACGCAAT | 119 | TATCAAAAAA | 120 |
| | | ATCGAAAAAC | | CCCCACACA | |
| | | CG | | ATCACCTC | |
| VCY | ENSG00000129864 | CCCACGCAAT | 121 | TATCAAAAAA | 122 |
| | | ATCGAAAAAC | | CCCCACACA | |
| | | CG | | ATCACCTC | |
| VCY | ENSG00000129864 | CCCACGCAAT | 123 | TATCAAAAAA | 124 |
| | | ATCGAAAAAC | | CCCCACACA | |
| | | CG | | ATCACCTC | |
| STOM | ENSG00000148175 | TCTAACGACC | 125 | AAACTTAACA | 126 |
| | | AAACTTAACA | | TCTAACAACC | |
| | | GCGAAATAA | | ACAAAATAA | |
| S1PR1 | ENSG00000170989 | AGTCGTATAG | 127 | AGTTGTATAG | 128 |
| | | ATTTCGGGTT | | ATTTTGGGTT | |
| | | TTTCGAACG | | TTTTGAATG | |
| SYNE1 | ENSG00000131018 | CTACGACCG | 129 | TCTCAACCCA | 130 |
| | | TCTCAACCCG | | CTACAACAAC | |
| | | AC | | | |
| ZNF304 | ENSG00000131845 | ACACGAACC | 131 | ACACAAACC | 132 |
| | | GAACATAAAA | | AAACATAAAA | |
| | | AAAATACGTC | | AAAATACATC | |
| ZNF304 | ENSG00000131845 | CCGACGTAC | 133 | CCAACATACA | 134 |
| | | AACCAATAAC | | ACCAATAACA | |
| | | AACCCAAAAA | | ACCCAAAAA | |
| | | C | | C | |
| SFMBT2 | ENSG00000198879 | CGAACCGAA | 135 | CAAACCAAAA | 136 |
| | | AAACTACTCT | | AACTACTCTC | |
| | | CCGACC | | CAACC | |
| SEPTIN9 | ENSG00000184640 | TGTTATGGGA | 137 | TGTTATGGGA | 138 |
| | | TTCGTATGTT | | TTTGTATGTT | |
| | | CGTTTTGCG | | TGTTTTGTG | |
| SEPTIN9 | ENSG00000184640 | CGTACGCGG | 139 | TGTATGTGGT | 140 |
| | | TTGGTAGGTA | | TGGTAGGTA | |
| | | GGATTT | | GGATTT | |
| SEPTIN9 | ENSG00000184640 | CGCGACCGC | 141 | CACAACCAC | 142 |
| | | AACAACCAAC | | AACAACCAA | |
| | | | | C | |

In embodiments, a data evaluation is carried out by determining the 1) absolute copy number per ml of isolated sample of the methylated tumour DNA sequences and 2) the absolute copy number per ml of isolated sample of the non-methylated DNA sequences (DNA background). The higher the absolute copy numbers of methylated or non-methylated DNA sequences per ml of isolated sample, the higher is the probability that there is a malignant tumour disease.

For each gene and the specific PCR conditions, the bias can be determined by a sample of known methylated DNA/non-methylated DNA ratio.

Since the bias is constant for each DNA sequence in the case of fixed PCR conditions, the relative frequencies of methylated DNA fragments between different samples can be compared with one another.

Absolute copy numbers of the methylated tumour DNA sequences and of the non-methylated DNA sequences (DNA background) per ml of isolated sample are determined depending on the selected PCR conditions in the amplification (step c)), which as a reference range (normal range, healthy) speak for the absence of a malignant disease. The limit values of the reference ranges (cut-off values) between healthy and sick are defined for each DNA sequence, and the PCR conditions are defined using extensive examinations of healthy subjects and patients with benign diseases.

In embodiments, patients having absolute copy numbers of methylated DNA and/or absolute copy numbers of non-methylated DNA per ml of isolated sample above the limit values for at least one gene, preferably at least two genes, particularly preferably at least three genes, selected from GSTP1, RASSF1A, CCDC181, NRIP3, miR129-2, SOX8, S1PR1, SYNE1, SFMBT2, ZNF304, or SEPTIN9, are classified as tumour patients.

In embodiments, a risk score analysis is performed after step d). The term “risk score analysis” is understood to mean a determination of the number of genes (biomarkers) selected from GSTP1, RASSF1A, CCDC181, NRIP3, miR129-2, SOX8, S1PR1, SYNE1, SFMBT2, ZNF304, or SEPTIN9, which show absolute copy numbers of methylated DNA or absolute copy numbers of non-methylated DNA per ml of isolated sample above the limit values.

In embodiments, the number of genes (biomarkers) with pathologically increased copy numbers of methylated tumour DNA sequences per ml of isolated sample is referred to as HexaPro score for prostate carcinoma or PentaRec score for colorectal carcinoma.

In preferred embodiments, the HexaPro score refers to the number of genes (biomarkers) selected from the genes GSTP1, RASSF1A, CCDC181, NRIP3, miR129-2, and SOX8, with pathologically increased copy numbers of methylated tumour DNA sequences per ml of isolated sample.

In preferred embodiments, the PentaRec score refers to the number of genes (biomarkers) selected from the genes S1PR1, SYNE1, CCDC181, SFMBT2, ZNF304, and SEPTIN9, with pathologically increased copy numbers of methylated tumour DNA sequences per ml of isolated sample.

In embodiments, a combination with at least one further biomarker selected from total PSA (tPSA), free PSA (fPSA), or fPSA/tPSA quotient (QfPSA), tPSA doubling time (tPSA-DT), carcinoembryonic antigen (CEA), cancer antigen 15-3 (CA 15-3), cancer antigen 125 (CA 125), the concentration of the isolated free-circulating DNA, and/or the amount of non-methylated DNA fragments of the corresponding gene sequences takes place.

In embodiments, the number of genes (biomarkers) with pathologically increased copy numbers of non-methylated DNA sequences per ml of isolated sample is referred to as U score (for prostate carcinoma and colorectal carcinoma).

In embodiments, HexaPro and PentaRec scores are combined with the corresponding U scores and with the results of further examinations in a multidimensional risk plot analysis. In embodiments, the combination is carried out with the determination of established biomarkers for the respective cancer, in particular the determination of the total PSA (tPSA, gene ID 354, UniProt ID P07288), free PSA (fPSA), or fPSA/tPSA quotient (QfPSA), and tPSA doubling time (tPSA-DT) for the prostate carcinoma or the determination of the carcinoembryonic antigen (CEA, gene ID 1048, UniProt ID P06731) in serum for colorectal carcinomas or the determination of cancer antigen 15-3 (CA 15-3, gene ID 4582, UniProt ID P15941) for breast cancer or the determination of cancer antigen 125 (CA 125, gene ID 94025, UniProt ID Q8WXI7) for ovarian cancer.

In embodiments for the detection and risk stratification of a PCa disease, patients are divided into 4 groups with increasing PCa risk (group I low risk, group IV highest risk) based on the HexaPro score (cut-off≤1) and the QfPSA value (cut-off≥20%, when using laboratory test kits by Roche or Abbott), with
Group I: HexaPro score≤1 and QfPSA≥20%,
Group II: HexaPro score≤1 and QfPSA<20%,
Group III: HexaPro score>1 and QfPSA≥20%, and
Group IV: HexaPro score>1 and QfPSA<20%.

Group I is associated with a low PCa risk in this embodiment. In embodiments, groups II-IV (higher PCa risk) are further subdivided as follows by the U score:
Subgroup A: U score≤1 (lower PCa risk) and
Subgroup B: U score>1 (higher PCa risk).

In embodiments, groups IIA/B-IVA/B are further subdivided using the tPSA-DT with a cut-off of tPSA-DT 10 months and by identifying the increased PCa risk for tPSA-DT<10 months with the * symbol. By combining the parameters mentioned, the following groups with an increasing PCa risk result: Groups I, IIA, IIA*, IIB, IIB*, IIIA, IIIA*, IIIB, IIIB*, IVA, IVA*, IVB and IVB*.

Advantageously, this multidimensional risk plot analysis provides a much more differentiated risk analysis for the presence of a PCa, an improvement in the indication of subsequent biopsy methods, diagnosis, prognosis, and therapy monitoring.

Another aspect of the invention relates to a computer program product comprising risk score analysis commands for diagnosing a tumour disease in an isolated sample, comprising
i. receiving data of the quantification of the methylated DNA sequences of at least four genes selected from GSTP1, RASSF1A, CCDC181, NRIP3, miR129-2, SOX8, S1PR1, SYNE1, SFMBT2, ZNF304, or SEPTIN9 in an isolated sample by means of digital PCR, obtained by means of the method according to the invention;
ii. determining the absolute copy numbers of methylated DNA per ml of isolated sample and/or of the percentage of methylated DNA sequences based on the total DNA sequences;
iii. comparison with limit values to distinguish healthy samples and patients with benign diseases from patients with malignant diseases, and to determine the risk score,
the risk score being the sum of the genes selected from GSTP1, RASSF1A, CCDC181, NRIP3, miR129-2, SOX8, S1PR1, SYNE1, SFMBT2, ZNF304, or SEPTIN9, which show absolute copy numbers of methylated and/or of the percentage of methylated DNA sequences based on the total DNA sequences above the limit values.

In embodiments, the computer program product comprises risk score analysis commands for diagnosing a tumour disease in an isolated sample, comprising receiving data of the quantification of the methylated and non-methylated DNA sequences in step a), and determining the absolute copy numbers of methylated and non-methylated DNA per ml of isolated sample and/or of the percentage of methylated DNA sequences based on the total DNA sequences in step b).

A further aspect of the invention relates to a data processing device, which comprises means for performing the method according to the invention comprising the risk score analysis and/or the computer program product according to the invention.

Another aspect of the invention relates to a kit for diagnosing a tumour disease in an isolated sample, comprising:
i) at least four primer pairs for the amplification of methylated DNA sequences for one gene, in each case, selected from GSTP1, RASSF1A, CCDC181, NRIP3, miR129-2, SOX8, S1PR1, SYNE1, SFMBT2, ZNF304, or SEPTIN9, by means of PCR,
the primer pairs each amplifying different genes, and
ii) at least one probe in each case for quantifying the amplified methylated DNA sequences of the at least four genes by means of digital PCR.

In embodiments, the kit comprises in component i) at least four primer pairs for amplifying methylated and non-methylated DNA sequences for one gene, in each case, selected from GSTP1, RASSF1A, CCDC181, NRIP3, miR129-2, SOX8, S1PR1, SYNE1, SFMBT2, ZNF304, or SEPTIN9, by means of PCR, the primer pairs each being suitable for amplifying methylated and non-methylated DNA sequences.

In embodiments, the kit comprises in component ii) at least one probe in each case for quantifying the amplified methylated and non-methylated DNA sequences of the at least four genes by means of digital PCR.

In embodiments, the kit comprises in component ii) at least one probe for quantifying methylated DNA and at least one further probe for quantifying non-methylated DNA.

In embodiments, the kit additionally contains primers for amplifying methylated and optionally non-methylated DNA sequences of at least one gene selected from TMEM106A, EYA4, GRIA4, ADAM32, VWA3B, ZNF833, ZNF529, USP44, HES5, ZFP37, PCSK9, RNF39, VCY, STOM, H2BC3, LONRF2, AKR1B1, HSPA1A, ZNF655, ZNF543, or GNE.

In embodiments, the primer pairs each have one to seven 5′-CG-3′ dinucleotides.

In embodiments, the probes for quantifying the amplified methylated DNA sequences each have two to eight 5′-CG-3′ or CpG dinucleotides.

In embodiments, the probes for quantifying the non-methylated DNA sequences each have two to eight 5′-CA-3′ or 5′-TG-3′ dinucleotides.

In embodiments, the kit furthermore comprises at least four primer pairs for amplifying methylated and optionally non-methylated DNA sequences for one gene, in each case, selected from GSTP1, RASSF1A, CCDC181, NRIP3, miR129-2, SOX8, S1PR1, SYNE1, SFMBT2, ZNF304, or SEPTIN9, by means of digital PCR in step d), the primer pairs each being suitable for amplifying methylated and non-methylated DNA sequences, the primer pairs each amplifying different genes.

In embodiments, the kit comprises extrinsic primers (as nested primers, component i) for step c) and intrinsic primers for step d).

In embodiments, the kit furthermore comprises at least one further component selected from a positive control, a negative control, an external standard, and a computer program product comprising risk score analysis commands for diagnosing a tumour disease in an isolated sample.

The term “positive control” is understood to mean a sample which provides a positive result during the error-free performance of the method and/or use of the kit. Advantageously, the positive control provides evidence that the method and/or kit have been correctly applied.

The term “negative control” is understood to mean a sample which provides a negative result during the error-free performance of the method and/or use of the kit.

In preferred embodiments, the negative control is a non-methylated DNA and the positive control is a methylated DNA.

In embodiments, DNA isolated from primary cells or cell lines is used as a positive control and/or negative control.

In embodiments, DNA from cells or cell lines in which the biomarkers (genes) are not methylated is used as a negative control. Preferably, DNA from epithelial cells of the healthy tissue corresponding to the tumour is used as a negative control. In embodiments, DNA from human prostate epithelial cells (PrEC) is used as a negative control for diagnosing prostate carcinomas. In embodiments, DNA from human mammary epithelial cells (HMEC) is used as negative control for diagnosing breast carcinomas. In alternative embodiments, DNA from MCF10A cell lines is used as a negative control for diagnosing breast carcinomas.

In embodiments, for each biomarker (gene), DNA selected from a cell line, in which the biomarker (gene) is present in homogeneously methylated form, such as U937, PC-3, DU-145, MCF-7, Cal-51, UACC-812, BT-474, MDA-MB-453, and/or MDA-MB231 cell lines, is used as a positive control for diagnosing tumour diseases.

In embodiments, for each biomarker (gene), DNA from a cell line, which has been isolated from a patient with breast or ovarian cancer, is used as a positive control for diagnosing breast or ovarian carcinomas.

The term “external standard” is understood to mean an aid in quantitative analyses for detecting sample losses, which is measured separately from the samples. An external standard advantageously allows for controlling the recovery of the DNA after isolation from an isolated sample (step a) and the bisulphite conversion (step b).

In embodiments, the external standard is a synthetic, double-stranded DNA sequence that must not occur in the human genome or the bisulphite-treated human genome, and the size distribution of which is similar to the fcDNA size distribution. The typical fragment length distribution of the fcDNA can be achieved by ligating the monomers of the external standard by means of T4 DNA ligase. The person skilled in the art can use commercially available kits for ligation.

In embodiments, the kit furthermore comprises a reaction buffer for amplification in step c), preferably with a magnesium chloride concentration in the range of 0.5 mmol/I to 15.0 mmol/I, preferably 2 mmol/I to 5 mmol/I, particularly preferably 2.5 mmol/I to 3.5 mmol/1, or a PCR buffer and a concentrated magnesium chloride solution, a reaction buffer for the quantification in step d), a desoxyribonucleotide mix, and/or a DNA polymerase, such as HotStarTaq Plus.

The invention also provides for using the kit according to the invention in order to carry out the method according to the invention.

A further aspect of the invention relates to the use of the method according to the invention and/or of the kit according to the invention for the diagnosis and/or progress control of a malignant tumour disease, in particular prostate, breast, ovarian, or colorectal carcinomas. The terms “diagnostics” and “progress control” within the meaning of the invention include, in particular, early diagnosis screening (prevention), prognosis, therapy control, and the detection of a minimal residual disease (MRD).

The detection of free-circulating tumour DNA using the method according to the invention can in principle be used for diagnosing any solid tumour, in particular when corresponding biomarkers (genes, “targets of interest”) are present in bodily fluids or smears (“liquid biopsies”).

The method according to the invention and the kit according to the invention are suitable in particular for diagnosing malignant tumours such as prostate, breast, ovarian, or colorectal carcinomas.

In embodiments, they are used for the early recognition of a malignant tumour disease, in particular prostate, breast, ovarian, or colorectal carcinomas. The method according to the invention and/or the kit according to the invention is advantageously suitable for diagnosing tumour diseases, in particular for early diagnosis, since individual cf tumour DNA copies are specifically amplified in the blood, urine, or other biological samples before a large background of “normal” wild-type DNA, before they are quantified by digital PCR.

In embodiments, the method according to the invention and/or the kit according to the invention is used in order to rule out a minimal residual disease (MRD), for the differential diagnosis of benign prostatic hyperplasia, prostatitis, and prostate carcinoma in an isolated sample, in particular at elevated PSA values or in the case of otherwise justified suspicion of a prostate carcinoma, or in order to diagnose breast cancer in an isolated sample, in particular in the case of ambiguous mammography results.

In embodiments, the method according to the invention and/or the kit according to the invention is used in combination with a PSA determination. It is expedient to use the method and/or kit according to the invention in the differential diagnosis of benign prostatic hyperplasia (BPH) and prostate carcinoma (PCa) diseases since the indication for a prostate tissue biopsy has to be established due to increased PSA values (critical range between 4.0 to 15.0 mg/ml, reference range 2.5 to 4.0 mg/ml).

In further embodiments, the method according to the invention and/or the kit according to the invention is used in combination with the PSA determination (tPSA, fPSA, QfPSA, tPSA-DT) and the indication for a tissue biopsy at elevated PSA values and the diagnosis of a prostate carcinoma, or in combination with mammography and suspicious results in the diagnosis of a breast carcinoma, or in combination with the presence of a gene mutation with increased familial risk for breast and ovarian carcinoma and the decision in favour of a prophylactic mastectomy and/or ovariectomy or for an improved indication of colonoscopy for suspected colorectal carcinoma.

In embodiments, tumour DNA is determined by means of the method according to the invention and/or by means of the kit according to the invention after surgery, chemotherapy, or radiotherapy, whereby the course of the disease is controlled and the presence of an MRD is diagnosed or ruled out. If no tumour DNA can be detected after therapy has taken place, a good response to the therapy can be assumed and an MRD ruled out. If tumour DNA is still detectable, therapy optimisation becomes necessary. If tumour DNA can be detected later on while it could not be detected earlier, a recurrence can be assumed and therapy optimisation becomes necessary.

In order to realise the invention, it is also expedient to combine the above-described embodiments according to the invention, the exemplary embodiments, and the features of the claims with one another.

### EXEMPLARY EMBODIMENTS

The invention is explained in more detail below with reference to an exemplary embodiment. The exemplary embodiment relates to . . . and is intended to describe the invention without limiting it.

The invention is explained in more detail with reference to drawings. In the drawings:

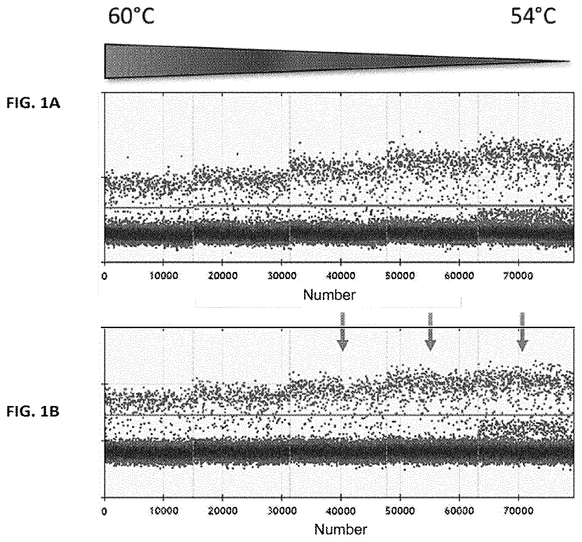

FIG. 1 shows the results of the dPCR detection system for the biomarker or the gene NRIP3. The fluorescence amplitudes of the (A) FAM and (B) HEX signals for methylated and non-methylated DNA sequences of the dPCR assay in the temperature gradient of 54 to 60° C. are shown.

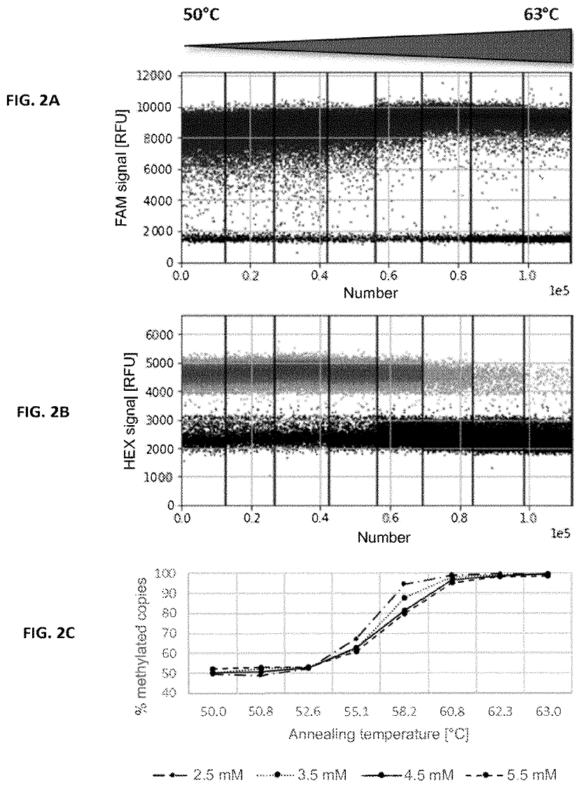

FIG. 2 shows the results of the dPCR detection system for the biomarker or the gene S1PR1 in the case of the variation of the magnesium chloride concentration and of the annealing temperature. Shown are the (A) FAM and (B) HEX signals as well as the percentage of methylated DNA sequences based on the total DNA sequences for the temperature gradient of 54 to 60° C. at a magnesium chloride concentration of 4.5 mM, and (C) the percentage of methylated DNA sequences based on the total DNA sequences as a function of the magnesium chloride concentration (2.5 to 5.5 mM) and of the temperature (50° C. to 63° C.).

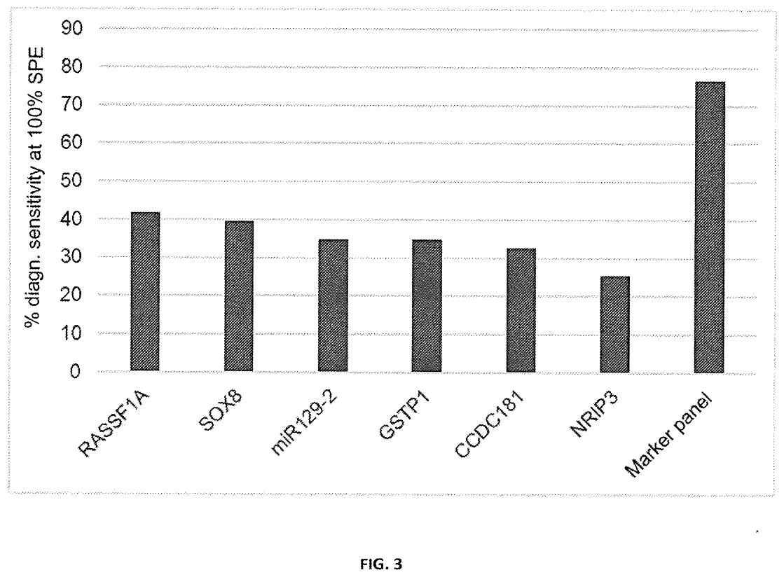

FIG. 3 shows the diagnostic sensitivity of the method and/or kit according to the invention for the PCa. Shown is the diagnostic sensitivity of the individual biomarkers (genes) RASSF1A, SOX8, miR129-2, GSTP1, CCDC181, and NRIP3, and of the marker panel (hexaplex reaction of the six biomarkers) at 100% specificity.

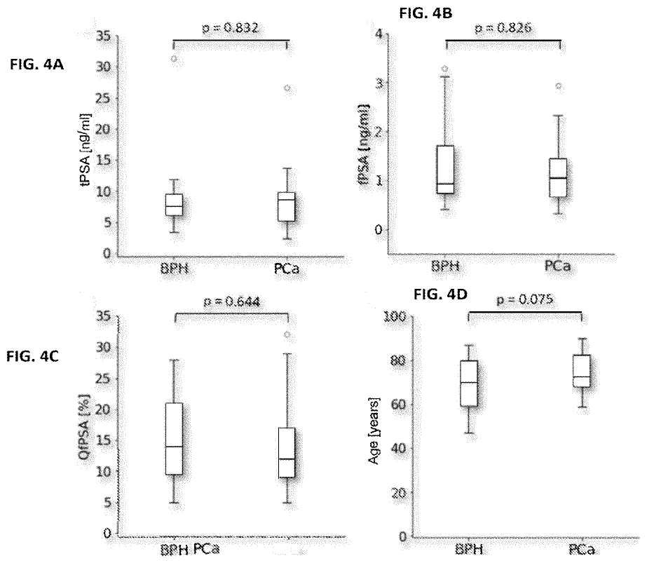

FIG. 4 shows an overview of the typical PCa risk factors total PSA (tPSA, A), free PSA (fPSA, B), and the fPSA/tPSA quotient (QfPSA, C) as well as the patient age (D) compared between the two groups of 24 BPH and 22 PCa patients.

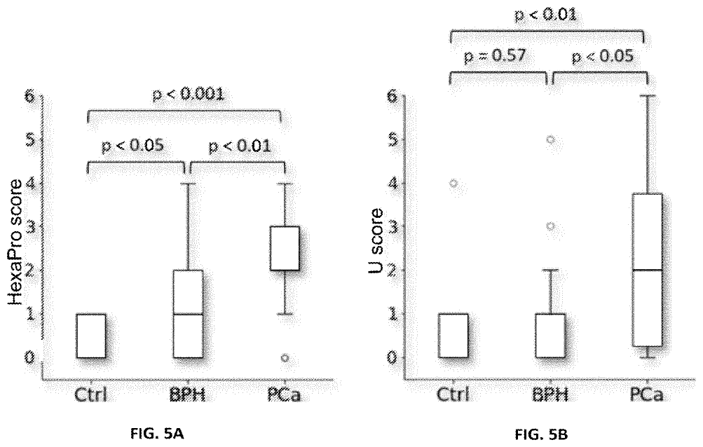

FIG. 5 shows the results of the methylation analysis of 33 healthy subjects (Ctrl) and 24 BPH and 22 PCa patients using the method according to the invention. The HexaPro score (A) or U score (B) was calculated from the sum total of 6 biomarkers for which an increased number of methylated (HexaPro score) or non-methylated (U score) DNA fragments per sample volume used was determined.

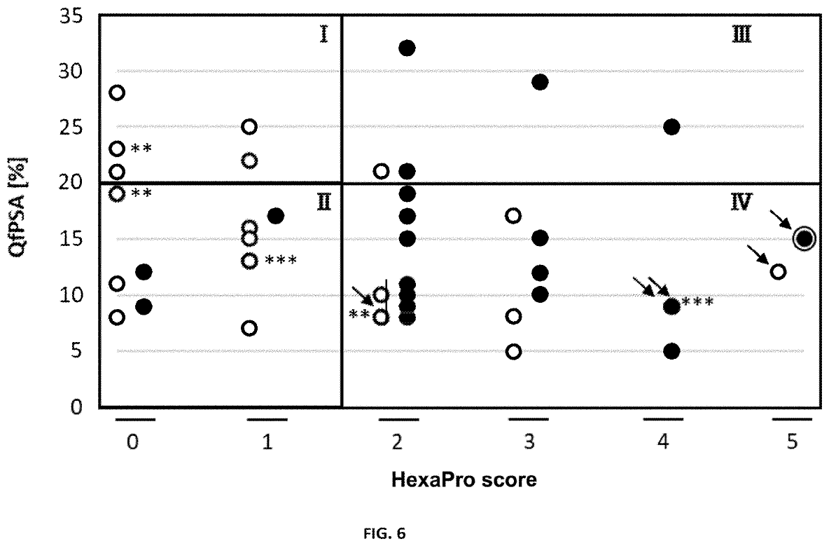

FIG. 6 shows a 2D classification of patients based on the HexaPro score and QfPSA value. To this end, 24 BPH patients (white) and 22 PCa patients (black) were investigated, and the HexaPro score and QfPSA value were plotted. The normal range cut-off for the HexaPro score was determined to be ≤1, and the normal range cut-off for QfPSA was determined to be ≥20% (when using laboratory test kits from Roche or Abbott). Region I: HexaPro negative and QfPSA negative; region II and III: only QfPSA positive or HexaPro positive; region IV: QfPSA positive and HexaPro positive. Overlapping data points with identical QfPSA and HexaPro value pairs are marked with ** (2 data points) or *** (3 data points). Patient samples in which hypermethylation of the GSTP1 marker was detected are marked with arrows.

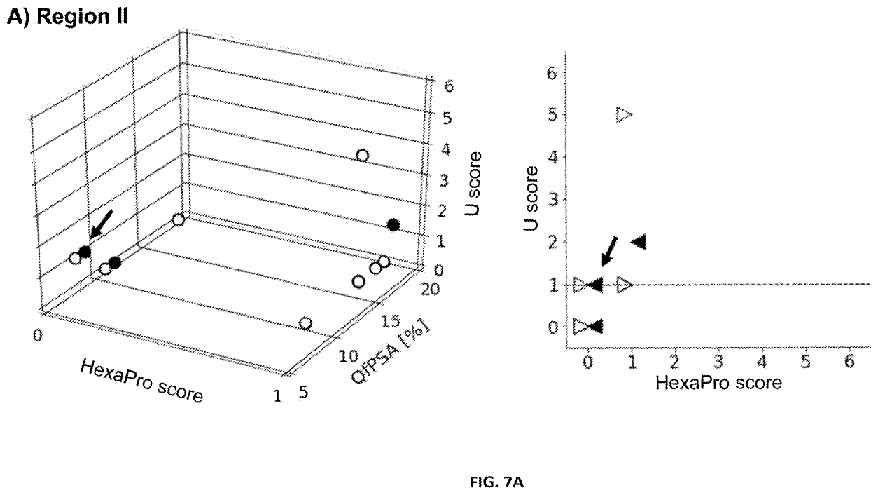

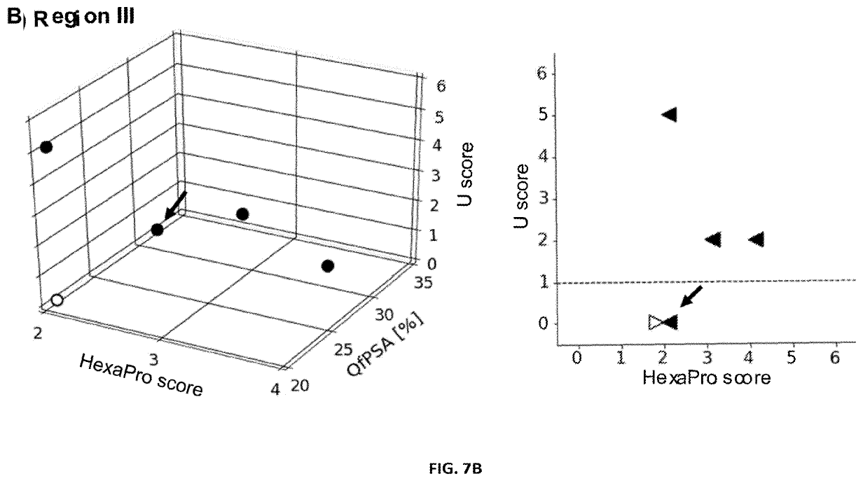

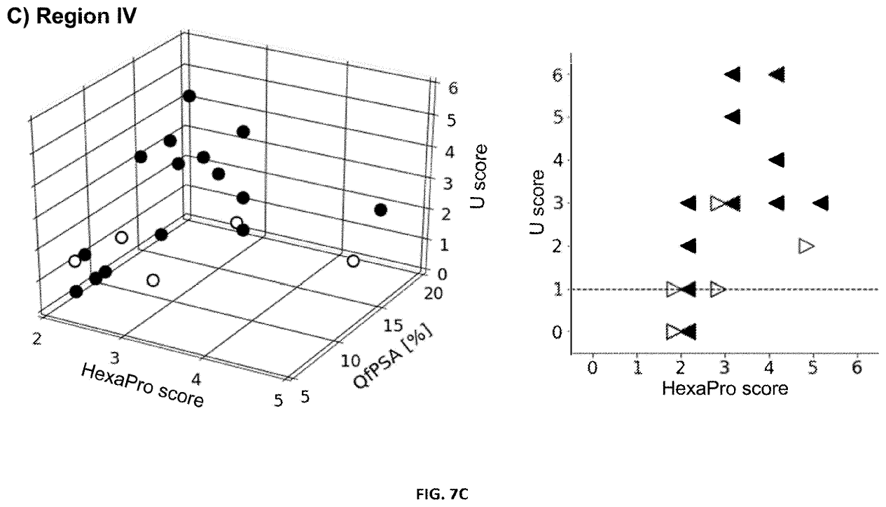

FIG. 7 shows a 3D classification of patients based on the HexaPro and U score and QfPSA value. For this purpose, 24 BPH patients (white) and 22 PCa patients (black) were examined, and the HexaPro value (x-axis) and QfPSA value (y-axis) and U score (z-axis) were plotted as 3D diagrams (left), and the HexaPro score (x-axis) and U score (z-axis) were plotted as 2D diagrams (right). The normal range cut-off for the HexaPro and U score was determined to be ≤1, and the normal range cut-off for QfPSA was determined to be ≥20%. Region II (A) or III (B): only QfPSA or HexaPro positive; region IV (C): QfPSA positive and HexaPro positive. Black arrows in regions II and III indicate two PCa patients whose PCa diagnosis was already established in TUR-P in 2016 and 2017, and who have been under “watchful waiting” treatment since then, without a distinct tPSA increase being recorded.

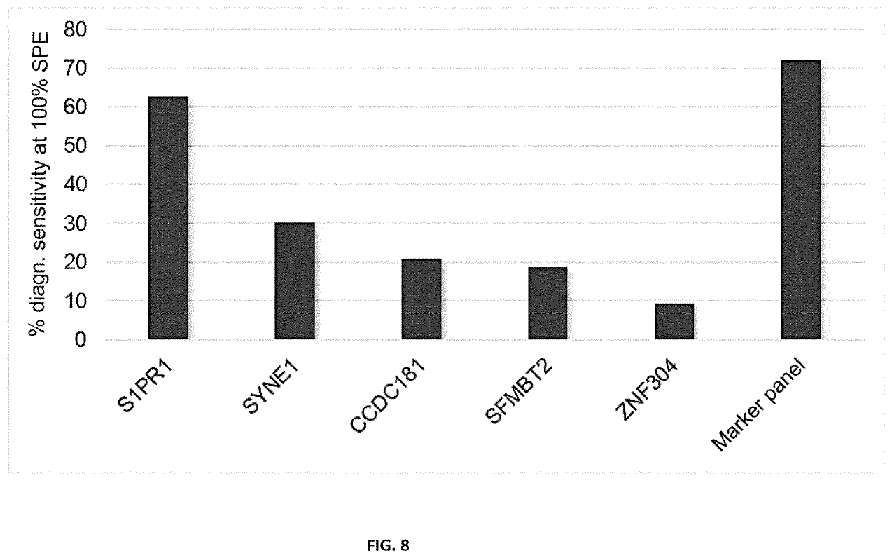

FIG. 8 shows the diagnostic sensitivity of the method and/or kit according to the invention for CRC. Shown is the diagnostic sensitivity of the individual biomarkers (genes) S1PR1, SYNE1, ZNF304, SFMBT2, and CCDC181, and of the marker panel (pentaplex reaction of the five biomarkers) at 100% specificity.

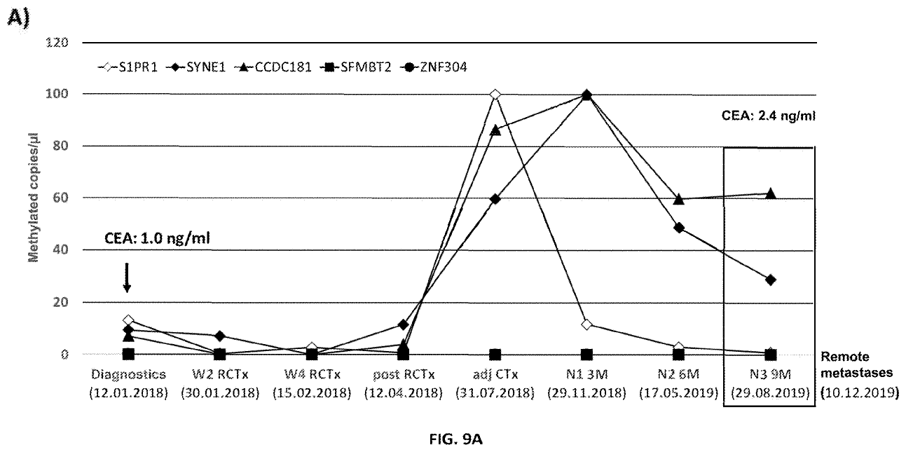

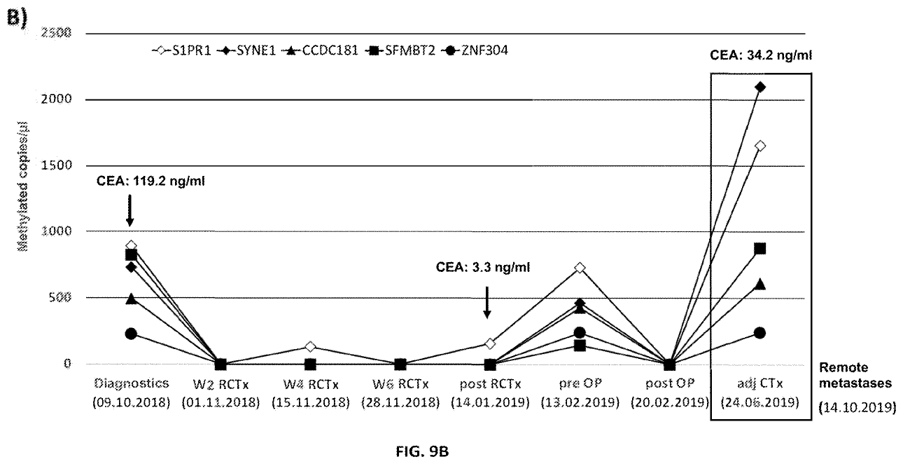

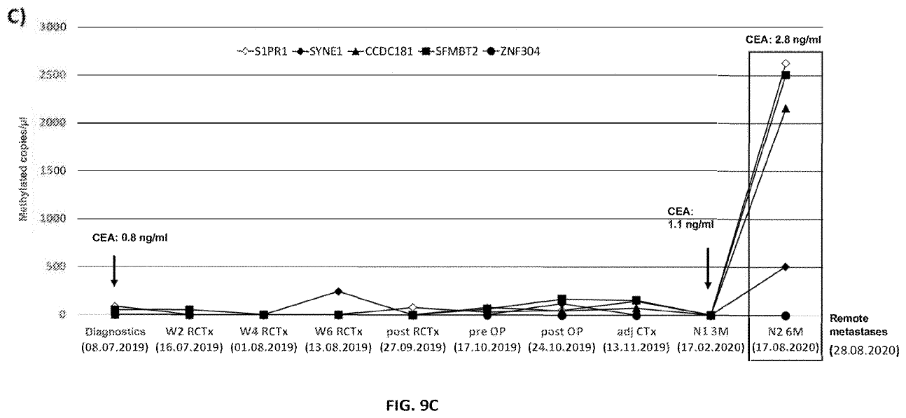

FIG. 9 shows the examination of CRC patients in the course of therapy using the method according to the invention. Shown are the results of the quantification of the methylated biomarkers (genes) S1PR1, SYNE1, CCDC181, SFMBT2, and ZNF304 with the method according to the invention using the example of three CRC therapy courses (A-C). Therapy protocol: week 2, 4 and 6 radio chemotherapy (W2-6 RCTx), after radio chemotherapy (post RCTx), before and after surgery (pre/post OP), adjuvant chemotherapy (adj CTx) and post-treatment appointments of all 3 months (N1-3). The normal range of CEA determination is <4.7 ng/ml (when using a laboratory test kit from Roche).

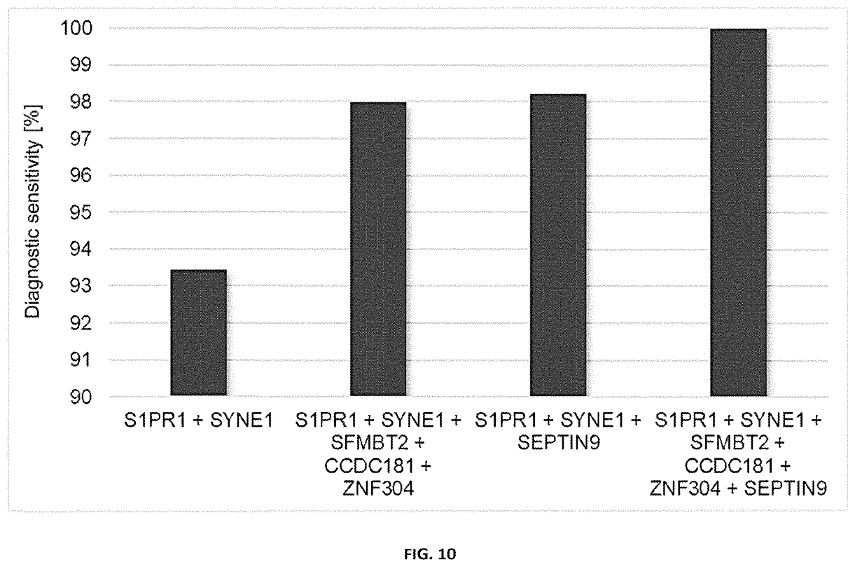

FIG. 10 shows the optimisation of the method according to the invention for the diagnosis of CRC by additional examination of the biomarker SEPTIN9. Shown are the diagnostic sensitivities for the analysis of CRC tumour and normal tissues (TCGA Research Network) at 100% specificity.

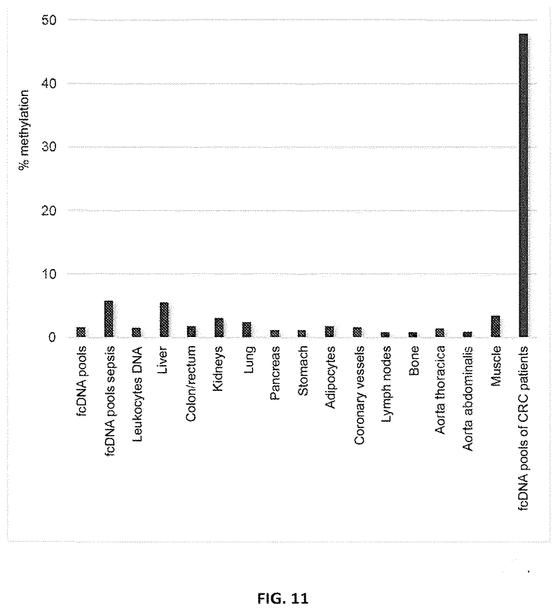

FIG. 11 shows the methylation status of SEPTIN9 for fcDNA pools of healthy subjects (n=5) and in fcDNA pools of CRC patients (n=4).

### GENERAL RULES

First, the free-circulating DNA (fcDNA) is isolated from the sample to be examined, in particular from 1-5 ml of blood serum or plasma samples using the QIAamp Circulating Nucleic Acid Kit from Qiagen GmbH (Hilden, FRG) according to the test kit description, and is eluted in 44 μl in each case.

The bisulphite conversion of 40 μl each of the fcDNA is carried out using the EpiTect Fast Bisulfite Conversion Kit from Qiagen GmbH according to the test kit description. After elution, 20 μl of bisulphite-treated fcDNA solution are obtained in each case.

Before and after bisulphite conversion, the DNA concentrations of the DNA samples are determined using the Quantus™ fluorometer (Promega). 1-4 μl of the bisulphite-treated DNA are used in step c).

TABLE 4
Reagents and their concentration for step c) (mM = mmol/l).

| Reagent | Volume [μl] | Final concentration | |
|---|---|---|---|
| PCR buffer [15 mM MgCl₂] | 2.50 | 1.5 | mmol/l |
| dNTPs [2.5 mM] | 2.00 | 200 | μmol/l |
| forward primer | 1.00 | 400 | nmol/l |
| reverse primer | 1.00 | 400 | nmol/l |
| HotStarTaq Plus [5 U/μl] | 0.125 | 0.625 | U |
| MgCl₂ [25 mM] | 0.00-8.00 | +0-8 | mmol/l |
| RNase-free aqua dest. | ad 25 μL | | |
| Carrier DNA | 1.00-4.00 | | |
| Final volume | 25.00 | | |

TABLE 5
Temperature and time scheme for step c).

| PCR | Temperature | Time |
|---|---|---|
| Denaturation | 95° C. | 5 minutes |
| PCR cycle | 94° C. (denaturation) | 10 seconds |
| 5 x-50 x | Primer-specific temperatures | 30 seconds |
| | (annealing: 50-72° C.) | 30 seconds |
| | 72° C. (elongation) | |
| | 4° C. | hold |

Testing of FAM- and HEX-labelled probes for the methylated or non-methylated biomarker (gene) NRIP3 is shown in FIG. 1.

Variation of the PCR reaction conditions (magnesium chloride concentration, annealing temperature) for step c) for the biomarker (gene) S1PR1 is shown in FIG. 2.

TABLE 6
Reagents and their concentration for step d).

| Reagent | Batch volume [μl] | Final concentration |
|---|---|---|
| 2x ddPCR Supermix (BioRad) | 10.0 | |
| 20x primer/probe mix | 1.0 | 900 mmol/l/250 nmol/l |
| DNA | 2.0 | |
| RNase-free aqua dest. | 7.0 | |
| Final volume | 20.0 | |

All probes (Table 6) were labelled 5′-FAM (methylated DNA) or 5′-HEX (non-methylated DNA) and with the quencher BHQ-1 at the 3′-end.

Production of the droplets (droplet generator [BioRad]) was carried out using 20 μl of master mix with DNA sample and 70 μl of oil in corresponding cartridges (BioRad), with about 20,000 oil/emulsion droplets being generated. 35 μl thereof were used in step d).

TABLE 7
Temperature and time scheme for step
d) using a T100 PCR device (BioRad).

| PCR | Temperature | Time |
|---|---|---|
| Denaturation | 95° C. | 10 minutes |
| PCR cycle | 94° C. (denaturation) | 30 seconds |
| 40 x | Primer- and probe-specific temperature | 1 minute |
| | (annealing and elongation, 50-72° C.) | |
| | 98° C. (stabilisation of the droplets) | 10 minutes |
| | 20° C. | hold |

Droplet fluorescence was measured by means of QX100 (BioRad) according to the manufacturer's description.

Reaction conditions for a cost- and time-saving multiplex reaction were investigated and determined based on the optimised individual reactions of the biomarkers in order to achieve increased sensitivity compared to the individual reactions (multiplex PCR, in particular pentaplex or hexaplex PCR). In order to better characterise the diagnostic specificity of the multiplex method according to the invention, blood samples from healthy subjects were examined. For the combination of various biomarkers, maximum values of the percentages of methylated fragments relative to the total template amount of the respective biomarker were determined as a limit value in order to ensure 100% diagnostic specificity. Diagnostic sensitivity was investigated with the previously defined parameters based on tumour patients at the time of diagnosis and in the course of the therapy.

### Examination of Prostate Carcinoma (PCa) Patients Using the Method According to the Invention

In order to examine prostate carcinoma (PCa) patients with the method according to the invention, the genes (biomarkers) RASSF1A, SOX8, miR129-2, GSTP1, CCDC181, and NRIP3 were analysed. In order to evaluate the diagnostic sensitivity and specificity, blood plasma samples from PCa patients (n=43, 56-89 years), patients with benign prostatic hyperplasia (BPH) (n=15, 50-73 years), and healthy subjects (n=52, 18-39 years) were examined. The single and hexaplex reactions (multiplex PCR of the six biomarkers) took place according to the oligonucleotide sequences and reaction conditions in Tables 1 to 3. For the biomarkers RASSF1A, SOX8, miR129-2, GSTP1, CCDC181, and NRIP3, it was possible to determine a sensitivity of 41.9% (RASSF1), 39.5% (SOX8), 65.1% (miR129-2), 34.9% (GSTP1), 32.6% (CCDC181), and 34.9% (NRIP3) at 100% diagnostic specificity (see FIG. 3). Moreover, the combination of the markers resulted in an increase in sensitivity to 76.7% at 100% specificity (or 90.7% sensitivity at 79.3% specificity) compared to the single-marker assays.

Compared to the PCa biomarkers according to the invention with 76% diagnostic sensitivity and 100% specificity, the typical tumour marker PSA has a relatively high diagnostic sensitivity of 91% but only a specificity of 14 to 21% (Rashid et al. 2012).

The method according to the invention was subsequently used in a prospective study with 46 patients with suspected PCa from whom blood was taken before a prostate tissue biopsy (PB). The tissue biopsies were performed due to suspicious PSA and/or DRU results and the question of whether a PCa is present in the patients. The age of the patients and the tPSA, fPSA, and QfPSA values (typical PCa markers) determined in the blood of the 24 BPH and 22 PCa patients, the diagnoses of which were histologically established based on the PB, are shown in FIG. 4 and do not differ significantly between the BPH and PCa patient groups.

The developed risk scores (HexaPro score of 0-6 based on the copy number per ml sample volume of the methylated and free-circulating tumour (fzT) DNA fragments, and U score of 0-6 points based on the copy number per ml sample volume of the non-methylated DNA background) showed very significant differences between the PCa group (n=22) and the control group that included healthy subjects (n=33) and the BPH group (n=24) (see FIG. 5).

In order to be able to better compare the individual values of the BPH and PCa patients with one another, a 2-dimensional plot consisting of QfPSA value and HexaPro score was prepared (see FIG. 6). In the resulting risk plot, region I (top left) contained measuring points from 6 (25%) of the 24 patients diagnosed with BPH in the prostate tissue biopsy (PB) but did not contain any measured value from patients with PCa. This group I was determined to have a low PCa risk. In contrast, region IV (bottom right) contained 15 PCa patients and, at 68%, the majority of the 22 PCa patients. Group IV was determined to have a high PCa risk. At the same time, 7 measuring points of patients with a PB-diagnosed BPH were in this region.

In region IV, there were 2 patients with GSTP1 hypermethylation in the BPH group and 3 patients with GSTP1 hypermethylation in the PCa group. Previous studies showed an increased risk of metastasis and a poorer prognosis for existing GSTP1 hypermethylation (Friedemann et al. 2021), which is why the HexaPro score was additionally increased by at least one point to increase the weighting of selective GSTP1 hypermethylation within the HexaPro score. In one patient of the PCa group, the diagnosis ASAP (atypical small acinar proliferation) was initially made based on a histological finding, while 4 of the 6 HexaPro markers (incl. GSTP1) were clearly positive (black circle). A PCa (Gleason score of 7) was found in this patient in a subsequent biopsy.

Region II (bottom left), on the other hand, contained measuring points of 10 BPH and 3 PCa patients, and region III (top right) contained measured values of 1 BPH and 4 PCa patients. Since there was a significantly higher number of BPH patients in region II and a smaller number of BPH patients in region III compared to PCa patients, it can be assumed that measured values in region II compared to III speak for a lower PCa risk and a higher weighting of the HexaPro score compared to QfPSA.

A solely QfPSA-based evaluation would have given an 82% diagnostic sensitivity (18 out of 22 PCa patients true positive) and a 29% specificity (7 out of 24 BPH patients true negative) based on a normal range cut-off of ≥20%. In comparison, with the aid of the HexaPro score and a normal range cut-off of ≤1, 4 PCa patients were identified that would have been overlooked when determining only the QfPSA, and the diagnostic sensitivity of the new score is 86% (19 out of 22 PCa patients true positive) at 100% specificity compared to the group of healthy subjects (33 out of 33 healthy subjects true negative) or a 67% specificity compared to the BPH group (16 out of 24 BPH patients true negative).

In order to achieve a further differentiation between BPH and PCa patients, in particular in groups II and III of the 2-D-HexaPro risk plot, we included the U score (based on the determination of the non-methylated DNA fragments in the method according to the invention) in the evaluation (see FIG. 7). Surprisingly, it was found before that the U score was much more sensitive compared to the fcDNA concentration, which we determined in parallel therewith (data not shown) and which has been often described as a potential biomarker for malignant diseases (Chen et al. 2021). This is why only the U score with a cut-off≤1 was used for the evaluations. Black arrows in regions II and III indicate two PCa patients whose PCa diagnosis was already established in TUR-P in 2016 and 2017, and who have been under “watchful waiting” treatment since then, without a distinct tPSA increase being recorded. The PSA-DT values were 29 and 26 months in these patients.

A point cloud with a U score>1 formed by 10 measuring points of the 15 PCa patients (67%) clearly emerged in particular in region IV of the 3D plot (FIG. 7C). This point cloud contained 5 measured values of PCa patients with a PSA-DT<10 months. It was also noteworthy that 2 of the 6 BPH patients in region IV also had a pathological U score and at the same time a PSA-DT of 7.9 and 4.3 months, wherein the latter patient sample was additionally marked by a GSTP1 positive result. There is numerous evidence that a GSTP1 positive result has prognostic significance with regard to the risk for cancer (Friedemann et al. 2021). It can therefore be concluded that these two BPH patients in region IV have a particularly high risk of a PCa and that there is a strong indication for repeating the biopsy in a timely manner (already after 3 months), which is less the case in the BPH patients whose measured values were in region II.

Region III of the 3D plot showed that the U scores of 3 of the 4 PCa patients were also pathologically increased and that the measuring points differed clearly from the other two measuring points in this region (FIG. 7B). Furthermore, 9 out of the 10 blood samples of the BPH patients in region II showed a negative U score (FIG. 7A). The following risk grouping was made based on these results:

TABLE 8
Risk grouping for prostate carcinoma.

| | Measured | Groups | | |
|---|---|---|---|---|
| Priority | variables | Designation | Limit values | PCa risk |
| 1 | HexaPro | Group I | QfPSA HexaPro score | low |
| | score & | Group II | ≥20%/≤1 | increased |
| | QfPSA value | Group III | <20%/≤1 | significantly increased |
| | | Group IV | ≥20%/>1 | high |
| | | | <20%/>1 | |
| 2 | U scores of | Subgroup A | U score | reduced risk |
| | groups II-IV | Subgroup B | ≤1 | increased risk . . . |
| | | | >1 | compared to priority 1 |
| 3 | PSA-DT | with * | PSA-DT | suggests possibly |
| | | without * | <10 months | higher aggressiveness |
| | | | ≥10 months | for PSA-DT <10 |
| | | | | months (*) |

If the individual patients are assigned to these groupings, the following distribution results which provides a much more differentiated risk analysis for the presence of a PCa:
Group I: 6 BPH patients
Group IIA: 9 BPH patients, 2 PCa patients
Group IIB: 1 BPH patient, 1 PCa patient
Group IIIA: 1 BPH patient, 1 PCa patient
Group IIIB: 3 PCa patients
Group IVA: 5 BPH patients, 5 PCa patients
Group IVB: 2 BPH patients, 10 PCa patients.

The problem with establishing a reliable BPH diagnosis that is to be defined in terms of time can be illustrated based on a case example in the cohort of this evaluation. Initially, a PCa Patient (FIG. 6, measuring point marked with a double circle in region IV) histologically showed an ASAP, i.e., no PCa, in the tissue biopsy performed immediately after the taking of a blood sample. In our 3D risk plot, however, we assigned this patient to the highest risk group IVb. In a new biopsy performed 3 months later, a PCa (GS 7a) was in the end detected, which was consistent with our risk grouping. 1 of the total of 3 PCa patients with a false negative result in the 3D-HexaPro plot (region IIA) was a patient whose PCa diagnosis by means of TUR-P (16 g with <5% and 12 g with <1% tumour cells) was already made in 2017, with him being under “watchful waiting” treatment ever since without showing a distinct increase in PSA. The PSA-DT value for this patient was 26 months (FIG. 7A, measuring point marked by an arrow).

As can be seen from the results, with the method according to the invention and the risk grouping I-IVB according to Table 8, a patient's risk for the presence of a PCa can be estimated much better than with the previous tumour markers PSA and QfPSA. In this way, unnecessary biopsies can be reduced and the correct diagnosis of a PCa can be significantly increased by an improved indication for a first tissue biopsy or repeat biopsy.

Examination of Patients with Colorectal Carcinoma (CRC) Using the Method According to the Invention

In order to examine patients with colorectal carcinoma (CRC) using the method according to the invention, genes (biomarkers) S1PR1, SYNE1, ZNF304, SFMBT2, and CCDC181 were analysed. The single and pentaplex reactions (multiplex PCR of the five biomarkers) took place according to the oligonucleotide sequences and reaction conditions in Tables 1 to 3. In order to evaluate the diagnostic sensitivity and specificity of the CRC marker panel, a training set consisting of 76 blood plasma samples from 17 CRC patients (CEA value in the range of 2.9-580.4 ng/ml) was first examined at the diagnosis time and in the course of the therapy. A series of 58 healthy subjects was analysed for comparison. A diagnostic sensitivity of 70.6% at 100% specificity was determined at the time of diagnosis. Then, 150 plasma samples from 26 CRC patients (CEA value in the range of 0.2-119 ng/ml) were analysed in a validation set at the time of diagnosis and in the course of the therapy. A diagnostic sensitivity of 73.1% at 100% specificity was determined at the time of diagnosis. With the method according to the invention, it was also possible to identify patients with CEA values in the normal range<4.7 ng/ml (when using a laboratory test kit from Roche) as being ill at the time of diagnosis. The diagnostic sensitivities of the individual biomarkers (genes) S1PR1, SYNE1, ZNF304, SFMBT2, and CCDC181, and of the marker panel (pentaplex reaction of the five biomarkers) of the training set and validation set are shown in FIG. 8. Compared to the method according to the invention for CRC with a diagnostic sensitivity of 72% (100% specificity), the sensitivity of the gold standard iFOBT (faecal immunochemical test) is significantly lower at 25% (90% specificity) in advanced adenomas. The determination of the CEA value also shows a lower sensitivity of 46% at 89% specificity.

Similarly to the analyses with the training set, a close relationship between the method according to the invention and the typical CRC parameter CEA was also found with the validation set, and important therapy events (chemotherapy cycles, surgery, recurrence/metastasis, etc.) were reflected by the number of methylated fragments of the biomarkers (genes) analysed (see FIG. 9). This suggests that the method according to the invention is suitable for therapy monitoring and stratification of the CRC disease.

Quantification of the methylated biomarkers (genes) S1PR1, SYNE1, CCDC181, SFMBT2, and ZNF304 with the method according to the invention using the example of three CRC therapy courses (A-C) is shown in FIG. 9. The normal range of CEA determination in serum is <4.7 ng/ml when using a laboratory test kit from Roche. The last measurement available before distant metastasis diagnosis is framed. (A) The biomarkers S1PR1, SYNE1, CCDC181 were positive at the time of diagnosis. The biomarkers SYNE1 and CCDC181 are significantly increased 4 months prior to the detection of distant metastases (leading time), while the CEA value is not. (B) All 5 biomarkers and CEA in serum are significantly increased at the time of diagnosis. Under therapy, methylation decreases again, before it increases again before surgery. After surgery, no methylated DNA fragments can be detected for any of the 5 markers. 4 months prior to distant metastasis diagnosis, all 5 markers reach maximum values, and the patient dies 5 months after the last blood examination. (C) For diagnosis, S1PR1 and SFMBT2 are slightly increased, and the CEA value in serum is normal. 11 days prior to distant metastasis diagnosis, a strong increase in the markers S1PR1, SYNE1, CCDC181, and SFMBT2 can be detected. The CEA value was in the normal range.

### Optimisation of the Method According to the Invention for Diagnosing CRC in Order to Maximise Diagnostic Sensitivity

In order to further improve the diagnostic sensitivity of the CRC marker panel, methylation data of CRC tumours were compared to the adjacent normal tissue (TCGA Research Network) and biomarkers were searched for that led to an increase in the diagnostic sensitivity of the pentaplex marker panel. It was possible to identify a region of the gene SEPTIN9 that contributed to an increase in diagnostic sensitivity, while specificity remained at the same level (see FIG. 10). The inclusion of SEPTIN9 as a biomarker in the method according to the invention results in an increase in the sensitivity of the CRC marker panel to 100%.

Furthermore, the degree of methylation of different regions of the SEPTIN9 gene (see Tables 1 and 3) was determined by means of different primers and probes. In order to characterise the background methylation of the regions examined, the methylation status of SEPTIN9 for pooled fcDNA samples from healthy subjects, genomic DNA of leukocytes, as well as a larger number of normal tissues, which qualify as a possible source of the fcDNA, was analysed. The results are shown in FIG. 11 and suggest a low basic methylation of SEPTIN9 in the blood. For comparison, the degree of methylation of SEPTIN9 in fcDNA pools of CRC patients (n=4) is shown, which, at an average degree of methylation of 48% (distinct hypermethylation), was significantly higher than the normal tissues analysed (fcDNA pools from healthy subjects, n=5) (methylation<6%).

Examination of Patients with Breast Cancer (MCa) or Ovarian Carcinoma (OCa) Using the Method According to the Invention

In order to examine breast cancer (MCa) or ovarian cancer patients with the method according to the invention, the genes (biomarkers) RASSF1A, GSTP1, CCDC181, and miR129-2 were analysed. The individual reactions took place according to the oligonucleotide sequences and reaction conditions in Tables 1 to 3. In order to evaluate the diagnostic sensitivity and specificity of the method according to the invention, 36 MCa patients with CA 15-3 values (tumour marker for breast cancer) in the range of 4.9 to 84.2 U were examined. While only 6 patients could be classified as being ill based on the CA 15-3 determination (cut-off CA 15-3<30U/I), increased percentages of methylated sequences were detectable in 28 patients (77.8% diagnostic sensitivity at 100% specificity) by means of the method according to the invention. 24 (80%) of the 30 breast cancer patients with normal CA 15-3 values (i.e., <30U/I) tested positive for the biomarkers RASSF1A, GSTP1, CCDC181, and miR129-2.

Moreover, 23 patients with an OCa diagnosis were examined. At the time of diagnosis, 16 patients had increased methylation levels for the biomarkers RASSF1A, GSTP1, CCDC181, and miR129-2 (69.6% diagnostic sensitivity at 100% specificity). The performance of the method according to the invention for MCa and OCa is summarised in Table 9.

TABLE 9
Diagnostic sensitivity and specificity of
the new marker panel for MCa and OCa.

| | | Diagnostic |
|---|---|---|
| Tumour entity | Diagnostic sensitivity [%] | specificity [%] |
| Breast cancer | 77.8% (28 out of 36 patients) | 100% |
| Ovarian | 69.6% (16 out of 23 patients) | 100% |
| carcinoma | | |

### CITED NON-PATENT LITERATURE

Chen E, Cario C L, Leong L, Lopez K, Merquez C P, Chu C, Li P S, Oropeza E, Tenggara I, Cowan J, Simko J P, Chan J M, Friedlander T, Wyatt A W, Aggarwal R, Paris P L, Carroll P R, Feng F Witte J S (2021) Cell-free DNA concentration and fragment size as a biomarker for prostate cancer. Scientific reports 11 (1), 5040.
Draht M X G, Goudkade D, Koch A, Grabsch H I, Weijenberg M P, van Engeland M, Melotte V, Smits K M (2018) Prognostic DNA methylation markers for sporadic colorectal cancer: a systematic review. Clin. Epigenetics 10 (35), 1-16.
Friedemann M, Horn F, Gutewort K, Tautz L, Jandeck C, Bechmann N, Sukocheva O, Wirth M P, Fuessel S, Menschikowski M (2021) Increased Sensitivity of Detection of RASSF1A and GSTP1 DNA Fragments in Serum of Prostate Cancer Patients: Optimisation of Diagnostics Using OBBPA-ddPCR. Cancers 13 (17), 4459.
Gritzmann R, Moinar B, Pilarsky C, Habermann J K, Schlag P M, Saeger H D, Miehlke S, Stolz T, Model F, Roblick U J, Bruch H P, Koch R, Liebenberg V, Devos T, Song X, Day R H, Sledziewski A Z, Lofton-Day C (2008) Sensitive detection of colorectal cancer in peripheral blood by septin 9 DNA methylation assay. PLoS one 3, e3759.
Luboldt H J, Fornara P, Weissbach L, Wirth M, Lorenz W, Rübben H. (2004) Systematic development of a guideline for early detection of prostate cancer: the German way in the evidence gap. Eur Urol. 46, 725-730.
Mikeska T, Candiloro I L, Dobrovic A (2010) Epigenomics. 2:561-73.
Roehl K A, Antenor J A V, Catalona W J (2002) Serial Biopsy Results in Prostate Cancer Screening Study. The Journal of Urology 167, 2435-2439.
Rashid M, Alam A, Habib A, Rahman H, Hossain A, Salam M Rahman S (2012) Efficacy of lower cut off value of serum prostate specific antigen in diagnosis of prostate cancer. Bangladesh Medical Research Council Bulletin 38 (3), 90-93.
Tao S, Hundt S, Haug U, Brenner H (2011) Sensitivity estimates of blood-based tests for colorectal cancer detection: impact of overrepresentation of advanced stage disease. Am J Gastroenterol. 106, 242-253.
Zaytoun O M, Stephenson A J, Fareed K, El-Shafei A, Gao T, Levy D, Jones J S (2012) When serial prostate biopsy is recommended: most cancers detected are clinically insignificant. BJU International 110, 987-992.

1. A method for diagnosing tumour disease in an isolated sample, comprising the steps of:
a) isolating DNA from an isolated sample;
b) bisulphite conversion of the DNA, wherein a conversion of non-methylated cytosine residues into uracil residues takes place;
c) amplifying methylated DNA sequences of at least four genes selected from GSTP1, RASSF1A, CCDC181, NRIP3, miR129-2, SOX8, SIPR1, SYNE1, SFMBT2, ZNF304, or SEPTIN9 by means of PCR;
d) quantifying the amplified methylated DNA sequences by means of digital PCR.

2. The method according to claim 1, wherein the isolated sample is a tissue sample, a bodily fluid, a faecal sample, or a smear.

3. The method according to claim 1, wherein, in step c), amplification of methylated DNA sequences
a. of the genes GSTP1, RASSF1A, CCDC181, NRIP3, miR129-2, and SOX8, or
b. of the genes SIPR1, SYNE1, CCDC181, SFMBT2, ZNF304, and SEPTIN9, or
c. of the genes GSTP1, RASSF1A, CCDC181, and miR129-2 takes place.

4. The method according to claim 1, wherein, in step c), the primer pair for the amplification of methylated DNA sequences is selected from
SEQ ID No. 23 and SEQ ID No. 24 for GSTP1,
SEQ ID No. 21 and SEQ ID No. 22 for RASSF1A,
SEQ ID No. 7 and SEQ ID No. 8 or SEQ ID No. 9 and SEQ ID No. 10 or SEQ ID No. 11 and SEQ ID No. 12 or SEQ ID No. 13 and SEQ ID No. 14 or SEQ ID No. 15 and SEQ ID No. 16 or SEQ ID No. 17 and SEQ ID No. 18 for CCDC181,
SEQ ID No. 27 and SEQ ID No. 28 or SEQ ID No. 29 and SEQ ID No. 30 or SEQ ID No. 31 and SEQ ID No. 32 or SEQ ID No. 33 and SEQ ID No. 34 or SEQ ID No. 35 and SEQ ID No. 36 for NRIP3,
SEQ ID No. 45 and SEQ ID No. 46 for miR129-2,
SEQ ID No. 39 and SEQ ID No. 40 or SEQ ID No. 41 and SEQ ID No. 42 for SOX8,
SEQ ID No. 69 and SEQ ID No. 70 for SIPR1,
SEQ ID No. 71 and SEQ ID No. 72 for SYNE1,
SEQ ID No. 85 and SEQ ID No. 86 or SEQ ID No. 87 and SEQ ID No. 88 for SFMBT2,
SEQ ID No. 75 and SEQ ID No. 76 or SEQ ID No. 77 and SEQ ID No. 78 for ZNF304, and
SEQ ID No. 93 and SEQ ID No. 94 or SEQ ID No. 95 and SEQ ID No. 96 or SEQ ID No. 97 and SEQ ID No. 98 for SEPTIN9.

5. The method according to claim 1, wherein, in step c), methylated DNA sequences of at least one gene selected from TMEM106A, EYA4, GRIA4, ADAM32, VWA3B, ZNF833, ZNF529, USP44, HES5, ZFP37, PCSK9, RNF39, VCY, STOM, H2BC3, LONRF2, AKR1B1, HSPA1A, ZNF655, ZNF543, or GNE are furthermore amplified and that, in step d), a quantification of the amplified methylated DNA sequences of the at least one gene is furthermore carried out by means of digital PCR.

6. The method according to claim 1, wherein the primer pairs used for amplification in step c) are used simultaneously in step d) during digital PCR.

7. The method according to claim 1, wherein quantification of the amplified DNA by means of digital PCR in step d) is carried out by means of probes, the probes having fluorescent markers and quenchers.

8. The method according to claim 1, wherein at least one further biomarker is furthermore determined.

9. The method according to claim 8, wherein the at least one further biomarker is selected from total PSA (tPSA), free PSA (fPSA) or fPSA/tPSA quotient (QfPSA), tPSA doubling time (tPSA-DT), carcinoembryonic antigen (CEA), cancer antigen 15-3 (CA 15-3), cancer antigen 125 (CA 125), the concentration of the isolated free-circulating DNA, and/or the amount of non-methylated DNA fragments of the corresponding gene sequences.

10. The method according to claim 1, wherein a risk score analysis is carried out after step d).

11. A computer program product comprising risk score analysis commands for diagnosing a tumour disease in an isolated sample, comprising
i) receiving data of the quantification of the methylated DNA sequences of at least four genes selected from GSTP1, RASSF1A, CCDC181, NRIP3, miR129-2, SOX8, SIPR1, SYNE1, SFMBT2, ZNF304, or SEPTIN9 in an isolated sample by means of digital PCR, obtained by means of a method according to claim 1;
ii) determining the absolute copy numbers of methylated DNA per ml of isolated sample and/or of the percentage of methylated DNA sequences based on the total DNA sequences;
iii) comparison with limit values to distinguish healthy samples and patients with benign diseases from patients with malignant diseases, and to determine the risk score,
the risk score being the sum of the genes selected from GSTP1, RASSF1A, CCDC181, NRIP3, miR129-2, SOX8, SIPR1, SYNE1, SFMBT2, ZNF304, or SEPTIN9, which show absolute copy numbers of methylated DNA per ml of isolated sample and/or of the percentage of methylated DNA sequences based on the total DNA sequences above the limit values.

12. The computer program product according to claim 11, wherein determination of the risk score takes place with the results of the determination of at least one further biomarker, preferably selected from total PSA (tPSA), free PSA (fPSA), or fPSA/tPSA quotient (QfPSA), tPSA doubling time (tPSA-DT), carcinoembryonic antigen (CEA), cancer antigen 15-3 (CA 15-3), cancer antigen 125 (CA 125), the concentration of the isolated free-circulating DNA, and/or the amount of non-methylated DNA fragments of the corresponding gene sequences.

13. A data processing device comprising means for performing the method according to claim 10.

14. A kit for diagnosing tumour disease in an isolated sample, comprising:
i. at least four primer pairs for the amplification of methylated DNA sequences for one gene, in each case, selected from GSTP1, RASSF1A, CCDC181, NRIP3, miR129-2, SOX8, SIPR1, SYNE1, SFMBT2, ZNF304, or SEPTIN9, by means of PCR,
the primer pairs each amplifying different genes, and
ii. at least one probe in each case for quantifying the amplified methylated DNA sequences of the at least four genes by means of digital PCR.

15. The kit according to claim 14, additionally comprising primers for amplifying methylated DNA sequences of at least one gene selected from TMEM106A, EYA4, GRIA4, ADAM32, VWA3B, ZNF833, ZNF529, USP44, HES5, ZFP37, PCSK9, RNF39, VCY, STOM, H2BC3, LONRF2, AKR1B1, HSPA1A, ZNF655, ZNF543, or GNE.

16. The kit according to claim 14, wherein the primer pairs each have one to seven 5′-CG-3′ dinucleotides and/or the probes for quantifying the amplified methylated DNA sequences each have two to eight 5′-CG-3′ dinucleotides.

17. The kit according to claim 14, further comprising at least one further component selected from a positive control, a negative control, an external standard, and a computer program product comprising risk score analysis commands for the diagnosis and risk stratification of a tumour disease in an isolated sample, said computer program product comprising
i) receiving data of the quantification of the methylated DNA sequences of at least four genes selected from GSTP1, RASSF1A, CCDC181, NRIP3, miR129-2, SOX8, SIPR1, SYNE1, SFMBT2, ZNF304, or SEPTIN9 in an isolated sample by means of digital PCR, obtained by means of a method for diagnosing tumour disease in an isolated sample, comprising the steps of:
a) isolating DNA from an isolated sample;
b) bisulphite conversion of the DNA, wherein a conversion of non-methylated cytosine residues into uracil residues takes place;
c) amplifying methylated DNA sequences of at least four genes selected from GSTP1, RASSF1A, CCDC181, NRIP3, miR129-2, SOX8, SIPR1, SYNE1, SFMBT2, ZNF304, or SEPTIN9 by means of PCR;
d) quantifying the amplified methylated DNA sequences by means of digital PCR;
ii) determining the absolute copy numbers of methylated DNA per ml of isolated sample and/or of the percentage of methylated DNA sequences based on the total DNA sequences;
iii) comparison with limit values to distinguish healthy samples and patients with benign diseases from patients with malignant diseases, and to determine the risk score,
the risk score being the sum of the genes selected from GSTP1, RASSF1A, CCDC181, NRIP3, miR129-2, SOX8, SIPR1, SYNE1, SFMBT2, ZNF304, or SEPTIN9, which show absolute copy numbers of methylated DNA per ml of isolated sample and/or of the percentage of methylated DNA sequences based on the total DNA sequences above the limit values.

18. A method according to claim 1 for the diagnosis and/or progress control of a malignant tumour disease.

19. The method according to claim 18 for the early diagnosis of a malignant tumour disease.

20. A method according to claim 18, wherein the malignant tumour disease is prostate, breast, ovarian, or colorectal carcinoma.


SEQUENCE LISTING (142 sequences)

SEQ ID NO: 1 - DNA, 23 bp, synthetic construct
  misc_feature 1..23: note = Forward Primer for TMEM106A
  source 1..23: mol_type = other DNA
agttttgatttagtgtttaggga

SEQ ID NO: 2 - DNA, 18 bp, synthetic construct
  misc_feature 1..18: note = Reverse Primer for TMEM106A
  source 1..18: mol_type = other DNA
tccgaaccctcgaataac

SEQ ID NO: 3 - DNA, 20 bp, synthetic construct
  misc_feature 1..20: note = Forward Primer for EYA4
  source 1..20: mol_type = other DNA
ggcgttattcgggatattta

SEQ ID NO: 4 - DNA, 18 bp, synthetic construct
  misc_feature 1..18: note = Reverse Primer for EYA4
  source 1..18: mol_type = other DNA
aaaacccactccgttttt

SEQ ID NO: 5 - DNA, 23 bp, synthetic construct
  misc_feature 1..23: note = Forward Primer for GRIA4
  source 1..23: mol_type = other DNA
ggatgagatatgcgttttatttt

SEQ ID NO: 6 - DNA, 23 bp, synthetic construct
  misc_feature 1..23: note = Reverse Primer for GRIA4
  source 1..23: mol_type = other DNA
ccgaactaatacaaacttactaa

SEQ ID NO: 7 - DNA, 23 bp, synthetic construct
  misc_feature 1..23: note = Forward Primer for CCDC181
  source 1..23: mol_type = other DNA
tcgttttcgtagttagagaggtt

SEQ ID NO: 8 - DNA, 24 bp, synthetic construct
  misc_feature 1..24: note = Reverse Primer for CCDC181
  source 1..24: mol_type = other DNA
cctcctctttctaaactcttcaca

SEQ ID NO: 9 - DNA, 22 bp, synthetic construct
  misc_feature 1..22: note = Forward Primer for CCDC181
  source 1..22: mol_type = other DNA
tggaggacgtagaggtacgttg

SEQ ID NO: 10 - DNA, 27 bp, synthetic construct
  misc_feature 1..27: note = Reverse Primer for CCDC181
  source 1..27: mol_type = other DNA
cctcctctttctaaactcttcacatta

SEQ ID NO: 11 - DNA, 23 bp, synthetic construct
  misc_feature 1..23: note = Forward Primer for CCDC181
  source 1..23: mol_type = other DNA
cgtagttagagaggttcggatgg

SEQ ID NO: 12 - DNA, 27 bp, synthetic construct
  misc_feature 1..27: note = Reverse Primer for CCDC181
  source 1..27: mol_type = other DNA
cctcctctttctaaactcttcacatta

SEQ ID NO: 13 - DNA, 22 bp, synthetic construct
  misc_feature 1..22: note = Forward Primer for CCDC181
  source 1..22: mol_type = other DNA
cgtagttagagaggttcggatg

SEQ ID NO: 14 - DNA, 23 bp, synthetic construct
  misc_feature 1..23: note = Reverse Primer for CCDC181
  source 1..23: mol_type = other DNA
ctcctctcctcctctttctaaac

SEQ ID NO: 15 - DNA, 23 bp, synthetic construct
  misc_feature 1..23: note = Forward Primer for CCDC181
  source 1..23: mol_type = other DNA
cggatggaggacgcagaggcacg

SEQ ID NO: 16 - DNA, 23 bp, synthetic construct
  misc_feature 1..23: note = Reverse Primer for CCDC181
  source 1..23: mol_type = other DNA
ctcctctcctcctctttctaaac

SEQ ID NO: 17 - DNA, 22 bp, synthetic construct
  misc_feature 1..22: note = Forward Primer for CCDC181
  source 1..22: mol_type = other DNA
cggatggaggacgcagaggcac

SEQ ID NO: 18 - DNA, 23 bp, synthetic construct
  misc_feature 1..23: note = Reverse Primer for CCDC181
  source 1..23: mol_type = other DNA
ctcctctcctcctctttctaaac

SEQ ID NO: 19 - DNA, 20 bp, synthetic construct
  misc_feature 1..20: note = Forward Primer for ADAM32
  source 1..20: mol_type = other DNA
gtattttagtcgcgtggttg

SEQ ID NO: 20 - DNA, 21 bp, synthetic construct
  misc_feature 1..21: note = Reverse Primer for ADAM32
  source 1..21: mol_type = other DNA
aaatccaacaacacaaaacga

SEQ ID NO: 21 - DNA, 22 bp, synthetic construct
  misc_feature 1..22: note = Forward Primer for RASSF1A
  source 1..22: mol_type = other DNA
gtttgttagcgtttaaagttag

SEQ ID NO: 22 - DNA, 18 bp, synthetic construct
  misc_feature 1..18: note = Reverse Primer for RASSF1A
  source 1..18: mol_type = other DNA
aatacgacccttcccaac

SEQ ID NO: 23 - DNA, 21 bp, synthetic construct
  misc_feature 1..21: note = Forward Primer for GSTP1
  source 1..21: mol_type = other DNA
atcgtagcggttttagggaat

SEQ ID NO: 24 - DNA, 20 bp, synthetic construct
  misc_feature 1..20: note = Reverse Primer for GSTP1
  source 1..20: mol_type = other DNA
tccccaacgaaacctaaaaa

SEQ ID NO: 25 - DNA, 20 bp, synthetic construct
  misc_feature 1..20: note = Forward Primer for VWA3B
  source 1..20: mol_type = other DNA
cgcgggttatagagatttag

SEQ ID NO: 26 - DNA, 23 bp, synthetic construct
  misc_feature 1..23: note = Reverse Primer for VWA3B
  source 1..23: mol_type = other DNA
catcccgctaataaataaaacaa

SEQ ID NO: 27 - DNA, 20 bp, synthetic construct
  misc_feature 1..20: note = Forward Primer for NRIP3
  source 1..20: mol_type = other DNA
ttacgttagtttaggggatg

SEQ ID NO: 28 - DNA, 20 bp, synthetic construct
  misc_feature 1..20: note = Reverse Primer for NRIP3
  source 1..20: mol_type = other DNA
ataacaaaaccgacgaaaaa

SEQ ID NO: 29 - DNA, 18 bp, synthetic construct
  misc_feature 1..18: note = Forward Primer for NRIP3
  source 1..18: mol_type = other DNA
ttagtttaggggatgcgg

SEQ ID NO: 30 - DNA, 19 bp, synthetic construct
  misc_feature 1..19: note = Reverse Primer for NRIP3
  source 1..19: mol_type = other DNA
ctcacccgataacaaaacc

SEQ ID NO: 31 - DNA, 20 bp, synthetic construct
  misc_feature 1..20: note = Forward Primer for NRIP3
  source 1..20: mol_type = other DNA
cggcgtggataggtgagtag

SEQ ID NO: 32 - DNA, 24 bp, synthetic construct
  misc_feature 1..24: note = Reverse Primer for NRIP3
  source 1..24: mol_type = other DNA
ccctaaactaacgtaaactctaaa

SEQ ID NO: 33 - DNA, 18 bp, synthetic construct
  misc_feature 1..18: note = Forward Primer for NRIP3
  source 1..18: mol_type = other DNA
cggcgtggataggtgagt

SEQ ID NO: 34 - DNA, 24 bp, synthetic construct
  misc_feature 1..24: note = Reverse Primer for NRIP3
  source 1..24: mol_type = other DNA
ccctaaactaacgtaaactctaaa

SEQ ID NO: 35 - DNA, 18 bp, synthetic construct
  misc_feature 1..18: note = Forward Primer for NRIP3
  source 1..18: mol_type = other DNA
cgtggataggtgagtagg

SEQ ID NO: 36 - DNA, 24 bp, synthetic construct
  misc_feature 1..24: note = Reverse Primer for NRIP3
  source 1..24: mol_type = other DNA
ccctaaactaacgtaaactctaaa

SEQ ID NO: 37 - DNA, 22 bp, synthetic construct
  misc_feature 1..22: note = Forward Primer for ZNF833
  source 1..22: mol_type = other DNA
cgtttgttaagggttgtgtttg

SEQ ID NO: 38 - DNA, 23 bp, synthetic construct
  misc_feature 1..23: note = Reverse Primer for ZNF833
  source 1..23: mol_type = other DNA
attaaatacgactccaaatcccg

SEQ ID NO: 39 - DNA, 19 bp, synthetic construct
  misc_feature 1..19: note = Forward Primer for SOX8
  source 1..19: mol_type = other DNA
gtaggttattcgggtttcg

SEQ ID NO: 40 - DNA, 20 bp, synthetic construct
  misc_feature 1..20: note = Reverse Primer for SOX8
  source 1..20: mol_type = other DNA
taactcgaatcctttcaacc

SEQ ID NO: 41 - DNA, 18 bp, synthetic construct
  misc_feature 1..18: note = Forward Primer for SOX8
  source 1..18: mol_type = other DNA
gggtcgtaagttcggaga

SEQ ID NO: 42 - DNA, 23 bp, synthetic construct
  misc_feature 1..23: note = Reverse Primer for SOX8
  source 1..23: mol_type = other DNA
aataactcgaatcctttcaacca

SEQ ID NO: 43 - DNA, 18 bp, synthetic construct
  misc_feature 1..18: note = Forward Primer for ZNF529
  source 1..18: mol_type = other DNA
ttttaggagttttcgggg

SEQ ID NO: 44 - DNA, 21 bp, synthetic construct
  misc_feature 1..21: note = Reverse Primer for ZNF529
  source 1..21: mol_type = other DNA
taataccaaccgcaataaaac

SEQ ID NO: 45 - DNA, 21 bp, synthetic construct
  misc_feature 1..21: note = Forward Primer for miR129-2
  source 1..21: mol_type = other DNA
acggtttggagaaatggagat

SEQ ID NO: 46 - DNA, 21 bp, synthetic construct
  misc_feature 1..21: note = Reverse Primer for miR129-2
  source 1..21: mol_type = other DNA
cgaaccctaaaaccgaacaaa

SEQ ID NO: 47 - DNA, 18 bp, synthetic construct
  misc_feature 1..18: note = Forward Primer for USP44
  source 1..18: mol_type = other DNA
gagaatttgtcggtggtc

SEQ ID NO: 48 - DNA, 20 bp, synthetic construct
  misc_feature 1..20: note = Reverse Primer for USP44
  source 1..20: mol_type = other DNA
acactacaaatacgaaaccc

SEQ ID NO: 49 - DNA, 19 bp, synthetic construct
  misc_feature 1..19: note = Forward Primer for HES5
  source 1..19: mol_type = other DNA
atatttcgcggttaatggt

SEQ ID NO: 50 - DNA, 18 bp, synthetic construct
  misc_feature 1..18: note = Reverse Primer for HES5
  source 1..18: mol_type = other DNA
aaaacgcccattataccc

SEQ ID NO: 51 - DNA, 21 bp, synthetic construct
  misc_feature 1..21: note = Forward Primer for ZFP37
  source 1..21: mol_type = other DNA
ttttgggataattagatcggg

SEQ ID NO: 52 - DNA, 23 bp, synthetic construct
  misc_feature 1..23: note = Reverse Primer for ZFP37
  source 1..23: mol_type = other DNA
atcaatcctataatacgtaacga

SEQ ID NO: 53 - DNA, 23 bp, synthetic construct
  misc_feature 1..23: note = Forward Primer for PCSK9
  source 1..23: mol_type = other DNA
gtcgtacgttttagtaagttttt

SEQ ID NO: 54 - DNA, 18 bp, synthetic construct
  misc_feature 1..18: note = Reverse Primer for PCSK9
  source 1..18: mol_type = other DNA
aatcgccgaaaactcttc

SEQ ID NO: 55 - DNA, 23 bp, synthetic construct
  misc_feature 1..23: note = Forward Primer for RNF39
  source 1..23: mol_type = other DNA
gtatatcgtcgtttgtttatttt

SEQ ID NO: 56 - DNA, 19 bp, synthetic construct
  misc_feature 1..19: note = Reverse Primer for RNF39
  source 1..19: mol_type = other DNA
aacgtccctaataaaacca

SEQ ID NO: 57 - DNA, 20 bp, synthetic construct
  misc_feature 1..20: note = Forward Primer for RNF39
  source 1..20: mol_type = other DNA
gtcgtagcgtataattggtt

SEQ ID NO: 58 - DNA, 18 bp, synthetic construct
  misc_feature 1..18: note = Reverse Primer for RNF39
  source 1..18: mol_type = other DNA
gcacccaacacaactaaa

SEQ ID NO: 59 - DNA, 21 bp, synthetic construct
  misc_feature 1..21: note = Forward Primer for VCY
  source 1..21: mol_type = other DNA
tttatatttggacggagaggg

SEQ ID NO: 60 - DNA, 19 bp, synthetic construct
  misc_feature 1..19: note = Reverse Primer for VCY
  source 1..19: mol_type = other DNA
ccccgaacactcaactaaa

SEQ ID NO: 61 - DNA, 21 bp, synthetic construct
  misc_feature 1..21: note = Forward Primer for VCY
  source 1..21: mol_type = other DNA
cgtgggtttgtttatatttgg

SEQ ID NO: 62 - DNA, 18 bp, synthetic construct
  misc_feature 1..18: note = Reverse Primer for VCY
  source 1..18: mol_type = other DNA
cccgaacactcaactaaa

SEQ ID NO: 63 - DNA, 22 bp, synthetic construct
  misc_feature 1..22: note = Forward Primer for VCY
  source 1..22: mol_type = other DNA
tgtttatatttggacggagagg

SEQ ID NO: 64 - DNA, 18 bp, synthetic construct
  misc_feature 1..18: note = Reverse Primer for VCY
  source 1..18: mol_type = other DNA
aaaccccgaacactcaac

SEQ ID NO: 65 - DNA, 18 bp, synthetic construct
  misc_feature 1..18: note = Forward Primer for STOM
  source 1..18: mol_type = other DNA
ttgaggttacgatggagg

SEQ ID NO: 66 - DNA, 23 bp, synthetic construct
  misc_feature 1..23: note = Reverse Primer for STOM
  source 1..23: mol_type = other DNA
acgaaataatacaaccctaacat

SEQ ID NO: 67 - DNA, 23 bp, synthetic construct
  misc_feature 1..23: note = Forward Primer for H2BC3
  source 1..23: mol_type = other DNA
ggcgttattttagtttgtttgtt

SEQ ID NO: 68 - DNA, 21 bp, synthetic construct
  misc_feature 1..21: note = Reverse Primer for H2BC3
  source 1..21: mol_type = other DNA
actttaactccattttccacg

SEQ ID NO: 69 - DNA, 21 bp, synthetic construct
  misc_feature 1..21: note = Forward Primer for S1PR1
  source 1..21: mol_type = other DNA
gggtagtagtaagatgcgaag

SEQ ID NO: 70 - DNA, 19 bp, synthetic construct
  misc_feature 1..19: note = Reverse Primer for S1PR1
  source 1..19: mol_type = other DNA
cctacctcgctcaaacaaa

SEQ ID NO: 71 - DNA, 19 bp, synthetic construct
  misc_feature 1..19: note = Forward Primer for SYNE1
  source 1..19: mol_type = other DNA
ggcgagttagttagggtga

SEQ ID NO: 72 - DNA, 23 bp, synthetic construct
  misc_feature 1..23: note = Reverse Primer for SYNE1
  source 1..23: mol_type = other DNA
cgccctaataacaaaaaacaaca

SEQ ID NO: 73 - DNA, 20 bp, synthetic construct
  misc_feature 1..20: note = Forward Primer for LONRF2
  source 1..20: mol_type = other DNA
gagtttcgtagggttgtaat

SEQ ID NO: 74 - DNA, 21 bp, synthetic construct
  misc_feature 1..21: note = Reverse Primer for LONRF2
  source 1..21: mol_type = other DNA
acctaaattacctaaaccgac

SEQ ID NO: 75 - DNA, 21 bp, synthetic construct
  misc_feature 1..21: note = Forward Primer for ZNF304
  source 1..21: mol_type = other DNA
cgttttttgttgttttgggtt

SEQ ID NO: 76 - DNA, 23 bp, synthetic construct
  misc_feature 1..23: note = Reverse Primer for ZNF304
  source 1..23: mol_type = other DNA
gatctctaaaactcaaccttcac

SEQ ID NO: 77 - DNA, 27 bp, synthetic construct
  misc_feature 1..27: note = Forward Primer for ZNF304
  source 1..27: mol_type = other DNA
ttgtagtttagagtagtaggaaacgtt

SEQ ID NO: 78 - DNA, 26 bp, synthetic construct
  misc_feature 1..26: note = Reverse Primer for ZNF304
  source 1..26: mol_type = other DNA
gttacccaaaaatctatacccaactc

SEQ ID NO: 79 - DNA, 21 bp, synthetic construct
  misc_feature 1..21: note = Forward Primer for AKR1B1
  source 1..21: mol_type = other DNA
tttttcgtttggttttagtgg

SEQ ID NO: 80 - DNA, 19 bp, synthetic construct
  misc_feature 1..19: note = Reverse Primer for AKR1B1
  source 1..19: mol_type = other DNA
gctttcccaccaaatacaa

SEQ ID NO: 81 - DNA, 21 bp, synthetic construct
  misc_feature 1..21: note = Forward Primer for HSPA1A
  source 1..21: mol_type = other DNA
ttcgaaaaaggtagtggattg

SEQ ID NO: 82 - DNA, 23 bp, synthetic construct
  misc_feature 1..23: note = Reverse Primer for HSPA1A
  source 1..23: mol_type = other DNA
cgacttataaaaacccaaaaaca

SEQ ID NO: 83 - DNA, 19 bp, synthetic construct
  misc_feature 1..19: note = Forward Primer for ZNF655
  source 1..19: mol_type = other DNA
ggaagcgggattattaggt

SEQ ID NO: 84 - DNA, 19 bp, synthetic construct
  misc_feature 1..19: note = Reverse Primer for ZNF655
  source 1..19: mol_type = other DNA
gccaacccatccttaaaac

SEQ ID NO: 85 - DNA, 23 bp, synthetic construct
  misc_feature 1..23: note = Forward Primer for SFMBT2
  source 1..23: mol_type = other DNA
gagggtaggacgaattttattag

SEQ ID NO: 86 - DNA, 18 bp, synthetic construct
  misc_feature 1..18: note = Reverse Primer for SFMBT2
  source 1..18: mol_type = other DNA
tactctccgaccaaccta

SEQ ID NO: 87 - DNA, 23 bp, synthetic construct
  misc_feature 1..23: note = Forward Primer for SFMBT2
  source 1..23: mol_type = other DNA
gggtaggacgaattttattaggt

SEQ ID NO: 88 - DNA, 18 bp, synthetic construct
  misc_feature 1..18: note = Reverse Primer for SFMBT2
  source 1..18: mol_type = other DNA
taaccaacgcactcaacc

SEQ ID NO: 89 - DNA, 22 bp, synthetic construct
  misc_feature 1..22: note = Forward Primer for ZNF543
  source 1..22: mol_type = other DNA
gtgggaatttattaagcgtagt

SEQ ID NO: 90 - DNA, 21 bp, synthetic construct
  misc_feature 1..21: note = Reverse Primer for ZNF543
  source 1..21: mol_type = other DNA
cctccgacttatacccataat

SEQ ID NO: 91 - DNA, 18 bp, synthetic construct
  misc_feature 1..18: note = Forward Primer for GNE
  source 1..18: mol_type = other DNA
gcggtagaggttatggaa

SEQ ID NO: 92 - DNA, 19 bp, synthetic construct
  misc_feature 1..19: note = Reverse Primer for GNE
  source 1..19: mol_type = other DNA
aattaactactcccgtccc

SEQ ID NO: 93 - DNA, 22 bp, synthetic construct
  misc_feature 1..22: note = Forward Primer for SEPTIN9
  source 1..22: mol_type = other DNA
ggagttggtggtttttcgttgg

SEQ ID NO: 94 - DNA, 23 bp, synthetic construct
  misc_feature 1..23: note = Reverse Primer for SEPTIN9
  source 1..23: mol_type = other DNA
actaacaaacaaaatcccgacct

SEQ ID NO: 95 - DNA, 22 bp, synthetic construct
  misc_feature 1..22: note = Forward Primer for SEPTIN9
  source 1..22: mol_type = other DNA
gtttgtaagggttaaacggtag

SEQ ID NO: 96 - DNA, 19 bp, synthetic construct
  misc_feature 1..19: note = Reverse Primer for SEPTIN9
  source 1..19: mol_type = other DNA
cctcgactcttaaacccac

SEQ ID NO: 97 - DNA, 19 bp, synthetic construct
  misc_feature 1..19: note = Forward Primer for SEPTIN9
  source 1..19: mol_type = other DNA
ggatttcgaaggtgggtgt

SEQ ID NO: 98 - DNA, 21 bp, synthetic construct
  misc_feature 1..21: note = Reverse Primer for SEPTIN9
  source 1..21: mol_type = other DNA
caaaatcctctccaacacgtc

SEQ ID NO: 99 - DNA, 27 bp, synthetic construct
  misc_feature 1..27: note = Probe for TMEM106A methylated
  source 1..27: mol_type = other DNA
aacgtctcaaactactacgtaacgaaa

SEQ ID NO: 100 - DNA, 27 bp, synthetic construct
  misc_feature 1..27: note = Probe for TMEM106A non-methylated
  source 1..27: mol_type = other DNA
aacatctcaaactactacataacaaaa

SEQ ID NO: 101 - DNA, 23 bp, synthetic construct
  misc_feature 1..23: note = Probe for EYA4 methylated
  source 1..23: mol_type = other DNA
cgtaaaaaccgcccaaaacctcg

SEQ ID NO: 102 - DNA, 23 bp, synthetic construct
  misc_feature 1..23: note = Probe for EYA4 non-methylated
  source 1..23: mol_type = other DNA
cataaaaaccacccaaaacctca

SEQ ID NO: 103 - DNA, 27 bp, synthetic construct
  misc_feature 1..27: note = Probe for CCDC181 methylated
  source 1..27: mol_type = other DNA
cccgacacctacctccgcgacaaccaa

SEQ ID NO: 104 - DNA, 27 bp, synthetic construct
  misc_feature 1..27: note = Probe for CCDC181 non-methylated
  source 1..27: mol_type = other DNA
cccaacacctacctccacaacaaccaa

SEQ ID NO: 105 - DNA, 23 bp, synthetic construct
  misc_feature 1..23: note = Probe for RASSF1A methylated
  source 1..23: mol_type = other DNA
cgcccaacgaataccaactcccg

SEQ ID NO: 106 - DNA, 23 bp, synthetic construct
  misc_feature 1..23: note = Probe for RASSF1A non-methylated
  source 1..23: mol_type = other DNA
cacccaacaaataccaactccca

SEQ ID NO: 107 - DNA, 20 bp, synthetic construct
  misc_feature 1..20: note = Probe for GSTP1 methylated
  source 1..20: mol_type = other DNA
cgcaacgaaatatacgcaac

SEQ ID NO: 108 - DNA, 20 bp, synthetic construct
  misc_feature 1..20: note = Probe for GSTP1 non-methylated
  source 1..20: mol_type = other DNA
cacaacaaaatatacacaac

SEQ ID NO: 109 - DNA, 21 bp, synthetic construct
  misc_feature 1..21: note = Probe for NRIP3 methylated
  source 1..21: mol_type = other DNA
cgacaatccgaaaatccaccg

SEQ ID NO: 110 - DNA, 22 bp, synthetic construct
  misc_feature 1..22: note = Probe for NRIP3 non-methylated
  source 1..22: mol_type = other DNA
caaccatctctaccactaacca

SEQ ID NO: 111 - DNA, 21 bp, synthetic construct
  misc_feature 1..21: note = Probe for SOX8 methylated
  source 1..21: mol_type = other DNA
cgtgtttcgcgttggttgttt

SEQ ID NO: 112 - DNA, 25 bp, synthetic construct
  misc_feature 1..25: note = Probe for SOX8 non-methylated
  source 1..25: mol_type = other DNA
tgtgttttgtgttggttgtttgatt

SEQ ID NO: 113 - DNA, 21 bp, synthetic construct
  misc_feature 1..21: note = Probe for SOX8 methylated
  source 1..21: mol_type = other DNA
cgtgtttcgcgttggttgttt

SEQ ID NO: 114 - DNA, 25 bp, synthetic construct
  misc_feature 1..25: note = Probe for SOX8 non-methylated
  source 1..25: mol_type = other DNA
tgtgttttgtgttggttgtttgatt

SEQ ID NO: 115 - DNA, 27 bp, synthetic construct
  misc_feature 1..27: note = Probe for miR129-2 methylated
  source 1..27: mol_type = other DNA
cgttccatcgcgactcaatctcaccac

SEQ ID NO: 116 - DNA, 27 bp, synthetic construct
  misc_feature 1..27: note = Probe for miR129-2 non-methylated
  source 1..27: mol_type = other DNA
cattccatcacaactcaatctcaccac

SEQ ID NO: 117 - DNA, 28 bp, synthetic construct
  misc_feature 1..28: note = Probe for HES5 methylated
  source 1..28: mol_type = other DNA
caatttaacgtacgctaataaaccgacg

SEQ ID NO: 118 - DNA, 28 bp, synthetic construct
  misc_feature 1..28: note = Probe for HES5 non-methylated
  source 1..28: mol_type = other DNA
caatttaacatacactaataaaccaaca

SEQ ID NO: 119 - DNA, 22 bp, synthetic construct
  misc_feature 1..22: note = Probe for VCY methylated
  source 1..22: mol_type = other DNA
atcgaaaaaccccacgcaatcg

SEQ ID NO: 120 - DNA, 27 bp, synthetic construct
  misc_feature 1..27: note = Probe for VCY non-methylated
  source 1..27: mol_type = other DNA
tatcaaaaaaccccacacaatcacctc

SEQ ID NO: 121 - DNA, 22 bp, synthetic construct
  misc_feature 1..22: note = Probe for VCY methylated
  source 1..22: mol_type = other DNA
atcgaaaaaccccacgcaatcg

SEQ ID NO: 122 - DNA, 27 bp, synthetic construct
  misc_feature 1..27: note = Probe for VCY non-methylated
  source 1..27: mol_type = other DNA
tatcaaaaaaccccacacaatcacctc

SEQ ID NO: 123 - DNA, 22 bp, synthetic construct
  misc_feature 1..22: note = Probe for VCY methylated
  source 1..22: mol_type = other DNA
atcgaaaaaccccacgcaatcg

SEQ ID NO: 124 - DNA, 27 bp, synthetic construct
  misc_feature 1..27: note = Probe for VCY non-methylated
  source 1..27: mol_type = other DNA
tatcaaaaaaccccacacaatcacctc

SEQ ID NO: 125 - DNA, 29 bp, synthetic construct
  misc_feature 1..29: note = Probe for STOM methylated
  source 1..29: mol_type = other DNA
aaacttaacatctaacgaccgcgaaataa

SEQ ID NO: 126 - DNA, 29 bp, synthetic construct
  misc_feature 1..29: note = Probe for STOM non-methylated
  source 1..29: mol_type = other DNA
aaacttaacatctaacaaccacaaaataa

SEQ ID NO: 127 - DNA, 29 bp, synthetic construct
  misc_feature 1..29: note = Probe for S1PR1 methylated
  source 1..29: mol_type = other DNA
agtcgtatagatttcgggtttttcgaacg

SEQ ID NO: 128 - DNA, 29 bp, synthetic construct
  misc_feature 1..29: note = Probe for S1PR1 non-methylated
  source 1..29: mol_type = other DNA
agttgtatagattttgggttttttgaatg

SEQ ID NO: 129 - DNA, 21 bp, synthetic construct
  misc_feature 1..21: note = Probe for SYNE1 methylated
  source 1..21: mol_type = other DNA
tctcaacccgctacgaccgac

SEQ ID NO: 130 - DNA, 20 bp, synthetic construct
  misc_feature 1..20: note = Probe for SYNE1 non-methylated
  source 1..20: mol_type = other DNA
tctcaacccactacaacaac

SEQ ID NO: 131 - DNA, 29 bp, synthetic construct
  misc_feature 1..29: note = Probe for ZNF304 methylated
  source 1..29: mol_type = other DNA
acacgaaccgaacataaaaaaaatacgtc

SEQ ID NO: 132 - DNA, 29 bp, synthetic construct
  misc_feature 1..29: note = Probe for ZNF304 non-methylated
  source 1..29: mol_type = other DNA
acacaaaccaaacataaaaaaaatacatc

SEQ ID NO: 133 - DNA, 30 bp, synthetic construct
  misc_feature 1..30: note = Probe for ZNF304 methylated
  source 1..30: mol_type = other DNA
ccgacgtacaaccaataacaacccaaaaac

SEQ ID NO: 134 - DNA, 30 bp, synthetic construct
  misc_feature 1..30: note = Probe for ZNF304 non-methylated
  source 1..30: mol_type = other DNA
ccaacatacaaccaataacaacccaaaaac

SEQ ID NO: 135 - DNA, 25 bp, synthetic construct
  misc_feature 1..25: note = Probe for SFMBT2 methylated
  source 1..25: mol_type = other DNA
cgaaccgaaaaactactctccgacc

SEQ ID NO: 136 - DNA, 25 bp, synthetic construct
  misc_feature 1..25: note = Probe for SFMBT2 non-methylated
  source 1..25: mol_type = other DNA
caaaccaaaaaactactctccaacc

SEQ ID NO: 137 - DNA, 29 bp, synthetic construct
  misc_feature 1..29: note = Probe for SEPTIN9 methylated
  source 1..29: mol_type = other DNA
tgttatgggattcgtatgttcgttttgcg

SEQ ID NO: 138 - DNA, 29 bp, synthetic construct
  misc_feature 1..29: note = Probe for SEPTIN9 non-methylated
  source 1..29: mol_type = other DNA
tgttatgggatttgtatgtttgttttgtg

SEQ ID NO: 139 - DNA, 25 bp, synthetic construct
  misc_feature 1..25: note = Probe for SEPTIN9 methylated
  source 1..25: mol_type = other DNA
cgtacgcggttggtaggtaggattt

SEQ ID NO: 140 - DNA, 25 bp, synthetic construct
  misc_feature 1..25: note = Probe for SEPTIN9 non-methylated
  source 1..25: mol_type = other DNA
tgtatgtggttggtaggtaggattt

SEQ ID NO: 141 - DNA, 19 bp, synthetic construct
  misc_feature 1..19: note = Probe for SEPTIN9 methylated
  source 1..19: mol_type = other DNA
cgcgaccgcaacaaccaac

SEQ ID NO: 142 - DNA, 19 bp, synthetic construct
  misc_feature 1..19: note = Probe for SEPTIN9 non-methylated
  source 1..19: mol_type = other DNA
cacaaccacaacaaccaac